SEQUENCE LISTING (3 sequences)

SEQ ID NO: 1 - DNA, 500 bp, Bacteriophage lambda
gatgagttcgtgtccgtacaactggcgtaatcatggcccttcggggccattgtttctctgtggaggagtccatgacgaaagatgaactgattgcccgtctccgctcgctgggtgaacaactgaaccgtgatgtcagcctgacggggacgaaagaagaactggcgctccgtgtggcagagctgaaagaggagcttgatgacacggatgaaactgccggtcaggacacccctctcagccgggaaaatgtgctgaccggacatgaaaatgaggtgggatcagcgcagccggataccgtgattctggatacgtctgaactggtcacggtcgtggcactggtgaagctgcatactgatgcacttcacgccacgcgggatgaacctgtggcatttgtgctgccgggaacggcgtttcgtgtctctgccggtgtggcagccgaaatgacagagcgcggcctggccagaatgcaataacgggaggcgctgtggctgatttcgataacc

SEQ ID NO: 2 - DNA, 25 bp, Artificial Sequence
  PCR primer
gatgagttcgtgtccgtacaactgg

SEQ ID NO: 3 - DNA, 24 bp, Artificial Sequence
  PCR primer
ggttatcgaaatcagcacagcgcc

(19) United States
(12) Patent Application Publication
(10) Pub. No.: US 2005/0239108 A1
(43) Pub. Date: Oct. 27, 2005

(54) Immuno-PCR method for the detection of a biomolecule in a test sample

(75) Inventors: Janet M. Barletta, Baltimore, MD (US); Niel T. Constantine, Baltimore, MD (US); Daniel Edelman, Baltimore, MD (US); Mark Manak, Laurel, MD (US); Jay Ji, Tucson, AZ (US); Chang-Chih Tai, Potomac, MD (US); William Highsmith JR., Rochester, MN (US)

Correspondence Address: SUGHRUE MION, PLLC, 2100 PENNSYLVANIA AVENUE, N.W., SUITE 800, WASHINGTON, DC, 20037, US

(73) Assignee: UNIVERSITY OF MARYLAND, BALTIMORE

(21) Appl. No.: 11/062,809

(22) Filed: Feb. 23, 2005

Related U.S. Application Data
(60) Provisional application No. 60/546,204, filed on Feb. 23, 2004.

(51) Int. Cl. C12Q 1/68

(52) U.S. Cl. 435/6

(57) ABSTRACT

The invention relates to methods and kits for detecting and/or monitoring biological molecules in a test sample. For example, the invention relates to methods and kits for detecting and/or monitoring HIV p24 antigen in human body fluid, biological toxins such as ricin or botulism in an environmental or biological sample, and prion protein from human, deer or bovine, such as PrP^{SC}, in a biological sample. The antigen detection signal is boosted by amplification of a polynucleotide linked to a detector molecule using methods for nucleic acid amplification technology.

The present application claims benefit of U.S. provisional application No. 60/546,204, filed Feb. 23, 2004, which is herein incorporated by reference.

### FIELD OF THE INVENTION

The invention relates to a method for detecting low levels of biomolecules in a sample. The biomolecules may be, e.g., of diagnostic or scientific significance, and include such biomolecules as proteins and peptides. Preferably, the proteins are medically important proteins such as the human immunodeficiency virus (HIV) p24 antigen, prion proteins such as human PrP^{SC}, deer PrP^{SC}, or bovine PrP^{SC}, and toxin such as ricin and botulinum toxin. The method may be used to detect biomolecules in biological samples such as blood, neuronal tissues, and urine, and in environmental samples such as water, soil, air and biological materials.

### BACKGROUND OF THE INVENTION

Since it was first identified in cattle in 1984, “mad cow disease,” also known as bovine spongiform encephalopathy (BSE), has been detected in about 200,000 cattle mostly in the United Kingdom and continental Europe. In humans, the clinical form of BSE is termed Creutzfeld-Jacob disease (CJD). The most common form is sporadic (sCJD) which is a rare, progressive, and fatal neurodegenerative disorder. Recently, a variant of CJD (vCJD) which occurs in young adults and has atypical clinical features and neuropathology has been described (Will et al., Lancet 347: 921-925; 1996). vCJD results from the consumption of tissue from cattle affected by bovine spongiform encephalopathy (BSE). Both BSE and vCJD are of significant economical concern, and a recent case of BSE in the United States has resulted in economically-devastating food bans and concerns for the safety of the blood supply.

The traditional diagnosis of BSE is based on histological examination of brain or lymphatic tissue. Most scientists believe that the humans who became infected did so after eating infected beef, and the concern that the disease may be spreading through the food supply and numerous consumer products derived from bovine parts has created a mushrooming market for an effective diagnostic test, preferably by testing live animals and humans. The lack of sensitive markers and the long latency period (5-10 years, or even decades) before an infected animal or human manifests symptoms make early disease diagnosis particularly difficult. Because of the lack of better diagnostic tests, more than 4.5 million cows, mainly in Britain, have been slaughtered on suspicion they have the disease, at an estimated cost of $2.5 billion. In the UK, the financial loss due to the 1996 ban on the export of beef is estimated at 5.5 billion dollars, and hundreds of thousands of cattle have been destroyed (Emerging Infectious Diseases 4:390-394 (1998)). In the US, the financial losses due to the ban on feed are about $200 million per year. Farmers in the US earn 54 billion dollars per year on meat and milk sales from 100 million cattle, plus 100 billion dollars from other industries (rendered protein). Therefore, the potential losses to US farmers could be 150 billion dollars per year if BSE infected cattle are detected in significant numbers.

Prion diseases of humans and animals are 100% fatal, there is no treatment available, and they cannot be diagnosed prior to the occurrence of clinical symptoms. At this point, consumption of the animal products could be a source of transmission. Because prion protein can be identified serologically only when present in high quantities in brain tissue, and because blood has been shown to be infectious (Brown et al., Transfusion 39, 1169-1178 (1999); Taylor et al., Journal of General Virology 77, 1595-1599 (1996), it is likely that the inability to detect prion protein in blood is due to a lack of sensitivity of current methods.

Although there have been no documented cases of CJD transmission through blood donation; animal studies have suggested that prions can be effectively transmitted through blood (Brown et al., Neurology 50, 684-688 (1998); Transfusion 39, 1169-1178 (1999)). Of critical importance is the question of how many people alive today might be harboring the abnormal prion protein that could be a source of infection through blood donation or organ/tissue transplantation. Central to the issue is that cases usually are not confirmed until after death, and therefore, blood being donated from these pre-symptomatic individuals may be capable of transmitting the infectious agent. The threat is compounded by the realization that one person's blood may be used as part of a pool to make thousands of blood products.

In the US, there have been 22 documented blood donors who subsequently developed CJD, with 322 persons receiving these products. This has led to recalls, quarantines, and donor deferrals (Sullivan, RAP Session Presentation, 52^{nd} Annual Meeting, AABB (1999)), and has resulted in shortages of protein derivatives such as immunoglobulin and alpha-1 protease inhibitor.

The medical community is reminded that during the early years of the AIDS epidemic, thousands of people who had transfusions became infected before blood-screening tests existed. With no blood screening test available for prion disease, the situation may be similar. In the US, millions of dollars worth of blood products have been destroyed due to the fear of CJD contamination, and at least 27 persons with sporadic CJD in the US have been infected through the use of human growth hormone for which the Department of Health has been found liable for compensation. Also, the threat of transmission through vaccines (e.g., measles) to children may pose a significant liability due to the widespread use of bovine products in their manufacture.

Thus, it is clear that prion diseases have an important impact on food safety, the animal industry, and blood safety.

There are several laboratory techniques that can be used to detect the abnormal prion protein and assist in the diagnosis of disease. However, all of these, with one exception, involve detection of PrP^{SC} (the abnormal form of prion) in tissue (brain or lymph tissue) using immunohistochemistry (the gold standard), ELISA, or the Western blot. Antigen capture ELISAs have been reported to detect prion protein in tissues (Zanusso, Proc. Natl. Acad. Sci. USA 95, 8812-8816 (1998)). Two ELISA methods (Bio-Rad Laboratories Hercules, Calif.; Enfer Scientific, Newbridge, Ireland) are approved for testing cattle brain material in Europe and one ELISA is approved in the US (IDEXX Laboratories, Westbrook, Me.) for detecting prion in deer.

Recently, there was a report of the detection of prion in urine after concentration using the Western blot (Shaked et al., Journal of Biological Chemistry 276, 31479-31482 (2001)), and a time resolved fluorescence immunoassay (TRFI) has been used to detect prion in tissues (Safar et al. Nature Medicine 4, 1157-1165 (1998)), but no method currently available for commercial use has been shown to detect prion in blood. Recently, Saborio et al. (Nature, 411: 810-813 (2001)) described a protein misfolding cyclic amplification method (PMCA) as an in vitro method to detect small amounts of abnormal prion in tissues by conversion of large amounts of normal prion. However, this method detected only 6-12 pg of prion, used the Western blot for detection, and required one day. The lone report of the ability to detect prion diseases from blood of animals (Schmerr et al., Journal of Chrom. A: 853:207-214 (1999)) using capillary electrophoresis (CE) has been met with controversy (Cervenakova et al., Transfusion 43:1687-1694 (2003).

The instant invention addresses the critical needs discussed above for a highly sensitive, accurate, and fast method of detecting very small quantities of biological molecules in a sample. Using a new technique that incorporates signal amplification and PCR, a simpler and less expensive detection assay is provided that offers ultra-sensitive detection, semi-quantitative measurements, and rapid turn-around time for results.

### SUMMARY OF THE INVENTION

Accordingly, an object of the present invention is to provide a simple and highly sensitive method for use, for example, in the detection of low levels of biomolecules in a test sample. For example, proteins and peptides that are of diagnostic or scientific significance may be detected in a test sample. Such proteins include viral antigens and prions that may be present in blood, neuronal tissue or urine at very low levels, and biowarfare reagents such as ricin and botulinum toxin that may be present in an environmental sample.

An additional object of the present invention is to provide a method that may be used in (1) the early diagnosis of BSE in cattle and deer, or vCJD in humans, (2) the determination of levels of human, bovine or deer PrP^{SC} in a sample, and (3) the monitoring of an individual's response to treatment of BSE or vCJD, through the testing of any human, bovine or deer body fluid such as plasma, serum, saliva, and whole blood for the presence of human, bovine or deer PrP^{SC}.

A further object of the present invention is to provide a kit comprising the elements needed to practice the methods described above.

The present invention has reached these goal by providing a modification of the technique disclosed in U.S. Pat. No. 5,665,539, with specific application for the detection of prion protein in biological or environmental samples.

In one embodiment, the above-described objects of the present invention have been met by a method for detecting a biomolecule in a sample. The method comprises:
(a) incubating a sample with a capture molecule that specifically binds a selected biomolecule under conditions such that said selected biomolecule in said sample is bound by said capture molecule, wherein said capture molecule comprises a molecule that specifically binds to said biomolecule and said capture molecule is attached to a solid support,
(b) incubating the resulting product of step (a) with a detector molecule that specifically binds said selected biomolecule under conditions such that said selected biomolecule bound by said capture molecule is also bound by said detector molecule, wherein said detector molecule comprises (i) a molecule that specifically binds to said biomolecule and (ii) at least one biotin moiety,
(c) incubating the resulting product of step (b) with a linker molecule under conditions such that said linker molecule binds to a biotin moiety of said detector molecule, wherein said linker molecule comprises at least one avidin moiety or at least one streptavidin moiety, or a combination thereof,
(d) incubating the resulting product of step (c) with an amplification reagent under conditions such that said amplification reagent binds to said linker molecule, wherein said amplification reagent comprises (i) a polynucleotide molecule and (ii) at least one biotin moiety,
(e) performing polymerase chain reaction (PCR) on the resulting product of step (d) in the presence of a signal molecule, and
(f) detecting a PCR product produced in step (e) by detecting a signal from the signal molecule, thereby detecting a biomolecule in said sample.

In a further embodiment, the above-described objects of the present invention have been met by a second method for detecting a biomolecule in a sample. The second method comprises:
(a) incubating a sample with a capture molecule that specifically binds a selected biomolecule under conditions such that said selected biomolecule in said sample is bound by said capture molecule, wherein said capture molecule comprises a molecule that specifically binds to said biomolecule and said capture molecule is attached to a solid support,
(b) incubating the resulting product of step (a) with a detector molecule that specifically binds said selected biomolecule under conditions such that said selected biomolecule bound by said capture molecule is also bound by said detector molecule, wherein said detector molecule comprises (i) a molecule that specifically binds to said biomolecule and (ii) at least one avidin moiety or at least one streptavidin moiety, or a combination thereof,
(c) incubating the resulting product of step (b) with an amplification reagent under conditions such that said amplification reagent binds to said avidin or said streptavidin, or both, of said detector molecule, wherein said amplification reagent comprises (i) a polynucleotide molecule and (ii) at least one biotin moiety,
(d) performing polymerase chain reaction (PCR) on the resulting product of step (c) in the presence of a signal molecule, and
(e) detecting a PCR product produced in step (d) by detecting a signal from the signal molecule, thereby detecting a biomolecule in said sample.

In an additional embodiment, the above-described objects of the present invention have been met in a third method for detecting a biomolecule in a sample. The third method comprises:
(A) incubating a sample with a capture molecule that specifically binds a selected biomolecule under conditions such that said selected biomolecule in said sample is bound by said capture molecule, wherein said capture molecule comprises a molecule that specifically binds to said biomolecule and said capture molecule is attached to a solid support,
(B) incubating the resulting product of step (A) with a detector-signal conjugate, said conjugate comprising
   (i) a detector molecule comprising (a) a molecule that specifically binds to said biomolecule and (b) at least one biotin moiety,
   (ii) a linker molecule bound to a biotin moiety of said detector molecule, wherein said linker molecule comprises at least one avidin moiety or at least one streptavidin moiety, or a combination thereof,
   (iii) an amplification reagent bound to an avidin moiety or a streptavidin moiety, or both, of said linker molecule, wherein said amplification reagent comprises (a) a polynucleotide molecule and (b) at least one biotin moiety,
(C) performing polymerase chain reaction (PCR) on the resulting product of step (B) in the presence of a signal molecule, and
(D) detecting a PCR product produced in step (C) by detecting a signal from the signal molecule, thereby detecting a biomolecule in a sample.

In an further additional embodiment, the above-described objects of the present invention have been met in a fourth method for detecting a biomolecule in a sample. The fourth method comprises:
(a) incubating a sample with a capture molecule that specifically binds a selected biomolecule under conditions such that said selected biomolecule in said sample is bound by said capture molecule, wherein said capture molecule comprises a molecule that specifically binds to said biomolecule and said capture molecule is attached to a solid support,
(b) incubating the resulting product of step (a) with a detector molecule-amplification reagent conjugate, said conjugate comprising a detector molecule attached to an amplification reagent, wherein said detector molecule comprises a molecule that specifically binds to said biomolecule and wherein said amplification reagent comprises a polynucleotide molecule,
(c) performing polymerase chain reaction (PCR) on the resulting product of step (b) in the presence of a signal molecule, and
(d) detecting a PCR product produced in step (c) by detecting a signal from the signal molecule, thereby detecting a biomolecule in a sample.

In a preferred embodiment, the above-described objects of the present invention have been met in a method for detecting PrP^{SC} in a sample. The method comprises:
(a) incubating a sample with a capture molecule that specifically binds PrP^{SC} under conditions such that PrP^{SC} in said sample is bound by said capture molecule, wherein said capture molecule comprises a molecule that specifically binds to PrP^{SC} and said capture molecule is attached to a solid support,
(b) incubating the resulting product of step (a) with a detector molecule that specifically binds PrP^{SC} under conditions such that PrP^{SC} bound by said capture molecule is also bound by said detector molecule, wherein said detector molecule comprises (i) a molecule that specifically binds to PrP^{SC} and (ii) at least one biotin moiety,
(c) incubating the resulting product of step (b) with a linker molecule under conditions such that said linker molecule binds to a biotin moiety of said detector molecule, wherein said linker molecule comprises at least one avidin moiety or at least one streptavidin moiety, or a combination thereof,
(d) incubating the resulting product of step (c) with an amplification reagent under conditions such that said amplification reagent binds to said linker molecule, wherein said amplification reagent comprises (i) a polynucleotide molecule and (ii) at least one biotin moiety,
(e) performing polymerase chain reaction (PCR) on the resulting product of step (d) in the presence of a signal molecule, and
(f) detecting a PCR product produced in step (e) by detecting a signal from the signal molecule, thereby detecting PrP^{SC} in said sample.

In preferred embodiments, PrP^{SC} is bovine PrP^{SC}, deer PrP^{SC} or human PrP^{SC}.

In a further embodiment, the above-described objects of the present invention have been met in a kit comprising the element necessary to practice the methods of the invention.

The kit comprises:
(a) a capture molecule attached to a solid support, wherein said capture molecule comprises a molecule that specifically binds to pre-selected biomolecule,
(b) a detector molecule, wherein said detector molecule comprises (i) a molecule that specifically binds to said pre-selected biomolecule and (ii) at least one biotin moiety,
(c) an linker molecule, wherein said linker molecule comprises at least one avidin moiety or at least one streptavidin moiety, or a combination thereof,
(d) an amplification reagent, wherein said amplification reagent comprises (i) a polynucleotide molecule and (ii) at least one biotin moiety,
(e) an oligonucleotide primer that specifically binds to said polynucleotide molecule,
(f) a signal molecule comprising (i) an oligonucleotide molecule that specifically binds the polynucleotide molecule, (ii) a fluorophore and (iii) a quencher dye, and
(g) a set of standards, wherein said standards allow an approximation of concentration of said pre-selected biomolecule in a sample.

In a further embodiment, the above-described objects of the present invention have been met in a second kit comprising the element necessary to practice the methods of the invention. The second kit comprises:
(a) a first capture molecule attached to a solid support, wherein said first capture molecule comprises a molecule that specifically binds to a pre-selected biomolecule,
(b) a second capture molecule attached to said solid support, wherein said second capture molecule comprises a molecule that specifically binds to an internal control molecule,
(c) an internal control molecule,
(d) a first detector molecule-amplification reagent conjugate, said conjugate comprising a first detector molecule attached to a first polynucleotide molecule, wherein said first detector molecule comprises a molecule that specifically binds to said pre-selected biomolecule,
(e) a second detector molecule-amplification reagent conjugate, said conjugate comprising a second detector molecule attached to a second polynucleotide molecule, wherein said second detector molecule comprises a molecule that specifically binds to said internal control molecule, and wherein said second polynucleotide molecule is different from said first polynucleotide molecule,
(f) a first oligonucleotide primer that specifically binds to said first polynucleotide molecule,
(g) a second oligonucleotide primer that specifically binds to said second polynucleotide molecule,
(h) a first signal molecule comprising (i) an oligonucleotide molecule that specifically binds the first polynucleotide molecule, (ii) a first fluorophore and (iii) a quencher dye,
(i) a second signal molecule comprising (i) an oligonucleotide molecule that specifically binds the second polynucleotide molecule, (ii) a second fluorophore that can be distinguish from said first fluorophore and (iii) a quencher dye,
(j) a first set of standards, wherein said first set of standards allows an approximation of concentration of said pre-selected biomolecule in a sample, and
(k) a second set of standards, wherein said second set of standards allows an approximation of concentration of said internal control molecule in a sample.

In still a further embodiment, the above-described objects of the present invention have been met in a third kit comprising the element necessary to practice the methods of the invention. The third kit comprises:
(a) a capture molecule attached to a solid support, wherein said capture molecule comprises a molecule that specifically binds to PrP^{SC},
(b) a detector molecule, wherein said detector molecule comprises (i) a molecule that specifically binds to PrP^{SC} and (ii) at least one biotin moiety,
(c) an linker molecule, wherein said linker molecule comprises at least one avidin moiety or at least one streptavidin moiety, or a combination thereof,
(d) an amplification reagent, wherein said amplification reagent comprises (i) a polynucleotide molecule and (ii) at least one biotin moiety,
(e) an oligonucleotide primer that specifically binds to said polynucleotide molecule,
(f) a signal molecule comprising (i) an oligonucleotide molecule that specifically binds the polynucleotide molecule, (ii) a fluorophore and (iii) a quencher dye, and
(g) a set of standards, wherein said standards allow an approximation of concentration of PrP^{SC} in a sample between about 3 attograms and 3 nanograms.

In still a further embodiment, the above-described objects of the present invention have been met in a fourth kit comprising the element necessary to practice the methods of the invention. The fourth kit comprises:
(a) a capture molecule attached to a solid support, wherein said capture molecule comprises a molecule that specifically binds to PrP^{SC},
(b) a detector molecule, wherein said detector molecule comprises (i) a molecule that specifically binds to PrP^{SC} and (ii) at least one avidin moiety,
(c) an amplification reagent, wherein said amplification reagent comprises (i) a polynucleotide molecule and (ii) at least one biotin moiety,
(d) an oligonucleotide primer that specifically binds to said polynucleotide molecule,
(e) a signal molecule comprising (i) an oligonucleotide molecule that specifically binds the polynucleotide molecule, (ii) a fluorophore and (iii) a quencher dye, and
(f) a set of standards, wherein said standards allow an approximation of concentration of PrP^{SC} in a sample between about 3 attograms and 3 nanograms.

### BRIEF DESCRIPTION OF THE DRAWINGS

FIG. 1 shows the four main steps of one embodiment of the invention: immunocapture, detection, amplification, and color production used to detect a biomolecule such as PrP^{SC}. The immunocapture step uses a capture antibody (capture molecule) specific for a prion, attached to a solid support to immobilize prions from a test sample. The detection step comprises the addition of different antibody that is biotinylated (the detector molecule), followed by the addition of a linker molecule (such as avidin or streptavidin) that binds to the biotinylated capture antibody. The amplification step comprises addition of an amplification reagent that binds to the linker molecule, where the amplification reagent comprises (i) a polynucleotide molecule and (ii) at least one biotin moiety. A signal molecule, comprising an oligonucleotide that specifically binds to the polynucleotide and having a fluorophore and quencher dye, is then added, followed by a PCR primer and necessary PCR reagents. PCR is then performed and the signal molecule is detected by the production of fluorescence for real-time analysis.

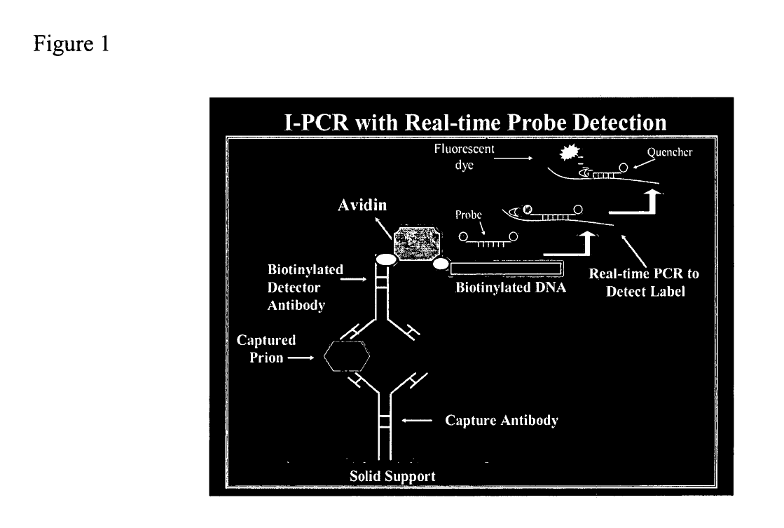

FIG. 2 shows the results of a comparison between an in-house ELISA and a SPIbio ELISA on PK-digested Normal and Scrapie Infected Hamster Brain Homogenates and Hamster Recombinant PrP^{C}. The in-house ELISA (using 7A12 or 8B4 as capture antibody) and the SPIbio ELISA were performed on dilutions of a 10% homogenate of normal or scrapie infected hamster brain homogenates digested with 50 ug/mL PK at 37° C. for 30 min. Positive samples for the in-house ELISA were defined as a signal to noise (S/N) of >2.0. The NSB threshold of the SPIbio ELISA was the mean OD of 4 replicates of NSB controls+3SD (0.114+0.009=0.123). Samples above this threshold were considered positive. Hamster recombinant PrP^{C} was used as a standard control and not PK-digested. All tests were performed in duplicate or greater replicates in multiple experiments. The mean OD of replicates is shown. {For all replicates, the standard errors (SEs) of the replicates were too small to display in the graph}.

FIG. 3 shows the results of tests performed to detect recombinant hamster prion by real-time IPCR. Concentrations of recombinant hamster prion are shown per mL. Zero controls are normal human plasma diluted 1:100 in lysis buffer.

FIG. 4 shows the results of tests performed to detect PK-treated normal and scrapie infected hamster brain. A 10% homogenate of normal or scrapie infected hamster brain was diluted 1:100 and treated with 50 ug/mL Proteinase K at 37° C. for 30 min. IPCR was performed on serial dilutions of the PK treated homogenate.

### DETAILED DESCRIPTION OF THE INVENTION

Generally, the methods of the present invention comprise application and binding of a capture molecule to a solid support, incubation of the capture molecule-bound support with a test sample containing a biomolecule to be detected, addition of a detector molecule, addition of a linker molecule, addition of an amplification reagent, performing PCR in the presence of a signal molecule, and real-time analysis of PCR products based on detection of signal.

In one embodiment, the present invention relates to a method for detecting a biomolecule in a sample. The method is practiced in an assay system through the following steps:
(a) incubating a sample with a capture molecule that specifically binds a selected biomolecule under conditions such that said selected biomolecule in said sample is bound by said capture molecule, wherein said capture molecule comprises a molecule that specifically binds to said biomolecule and said capture molecule is attached to a solid support,
(b) incubating the resulting product of step (a) with a detector molecule that specifically binds said selected biomolecule under conditions such that said selected biomolecule bound by said capture molecule is also bound by said detector molecule, wherein said detector molecule comprises (i) a molecule that specifically binds to said biomolecule and (ii) at least one biotin moiety,
(c) incubating the resulting product of step (b) with a linker molecule under conditions such that said linker molecule binds to a biotin moiety of said detector molecule, wherein said linker molecule comprises at least one avidin moiety or at least one streptavidin moiety, or a combination thereof,
(d) incubating the resulting product of step (c) with an amplification reagent under conditions such that said amplification reagent binds to said linker molecule, wherein said amplification reagent comprises (i) a polynucleotide molecule and (ii) at least one biotin moiety,
(e) performing polymerase chain reaction (PCR) on the resulting product of step (d) in the presence of a signal molecule, and
(f) detecting a PCR product produced in step (e) by detecting a signal from the signal molecule, thereby detecting a biomolecule in said sample.

An alternative method for detecting a biomolecule in a sample is also provided. The alternative method comprises:
(a) incubating a sample with a capture molecule that specifically binds a selected biomolecule under conditions such that said selected biomolecule in said sample is bound by said capture molecule, wherein said capture molecule comprises a molecule that specifically binds to said biomolecule and said capture molecule is attached to a solid support,
(b) incubating the resulting product of step (a) with a detector molecule that specifically binds said selected biomolecule under conditions such that said selected biomolecule bound by said capture molecule is also bound by said detector molecule, wherein said detector molecule comprises (i) a molecule that specifically binds to said biomolecule and (ii) at least one avidin moiety or at least one streptavidin moiety, or a combination thereof,
(c) incubating the resulting product of step (b) with an amplification reagent under conditions such that said amplification reagent binds to said avidin or said streptavidin, or both, of said detector molecule, wherein said amplification reagent comprises (i) a polynucleotide molecule and (ii) at least one biotin moiety,
(d) performing polymerase chain reaction (PCR) on the resulting product of step (c) in the presence of a signal molecule, and
(e) detecting a PCR product produced in step (d) by detecting a signal from the signal molecule, thereby detecting a biomolecule in said sample.

A further alternative method for detecting a biomolecule in a sample is provided. The further alternative method comprises:
(A) incubating a sample with a capture molecule that specifically binds a selected biomolecule under conditions such that said selected biomolecule in said sample is bound by said capture molecule, wherein said capture molecule comprises a molecule that specifically binds to said biomolecule and said capture molecule is attached to a solid support,
(B) incubating the resulting product of step (A) with a detector-signal conjugate, said conjugate comprising
   (i) a detector molecule comprising (a) a molecule that specifically binds to said biomolecule and (b) at least one biotin moiety,
   (ii) a linker molecule bound to a biotin moiety of said detector molecule, wherein said linker molecule comprises at least one avidin moiety or at least one streptavidin moiety, or a combination thereof,
   (iii) an amplification reagent bound to an avidin moiety or a streptavidin moiety, or both, of said linker molecule, wherein said amplification reagent comprises (a) a polynucleotide molecule and (b) at least one biotin moiety,
(C) performing polymerase chain reaction (PCR) on the resulting product of step (B) in the presence of a signal molecule, and
(D) detecting a PCR product produced in step (C) by detecting a signal from the signal molecule, thereby detecting a biomolecule in a sample.

An additional alternative method for detecting a biomolecule in a sample is provided. The further alternative method comprises:
(a) incubating a sample with a capture molecule that specifically binds a selected biomolecule under conditions such that said selected biomolecule in said sample is bound by said capture molecule, wherein said capture molecule comprises a molecule that specifically binds to said biomolecule and said capture molecule is attached to a solid support,
(b) incubating the resulting product of step (a) with a detector molecule-amplification reagent conjugate, said conjugate comprising a detector molecule attached to an amplification reagent, wherein said detector molecule comprises a molecule that specifically binds to said biomolecule and wherein said amplification reagent comprises a polynucleotide molecule,
(c) performing polymerase chain reaction (PCR) on the resulting product of step (b) in the presence of a signal molecule, and
(d) detecting a PCR product produced in step (c) by detecting a signal from the signal molecule, thereby detecting a biomolecule in a sample.

In a preferred embodiments, the present invention relates to a method for detecting human PrP^{SC}, deer PrP^{SC}, or bovine PrP^{SC} in a sample.

These embodiments provide advantages in the detection of low levels of PrP^{SC} in a sample through (1) higher sensitivity than is attainable by current PrP^{SC} ELISA methods, and (2) a cost-effective semi-quantitative serologic-molecular method.

The method of the preferred embodiment is practiced in an assay system through the following steps:
(a) incubating a sample with a capture molecule, where the capture molecule specifically recognizes and binds to PrP^{SC} that may be present in the sample and where the capture molecule is attached to a solid support,
(b) adding a detector antibody that specifically binds PrP^{SC} under conditions such that PrP^{SC} bound by the capture antibody is also bound by the detector antibody, where the detector antibody comprises at least one biotin moiety,
(c) adding a linker molecule under conditions such that the linker molecule binds to a biotin moiety of the detector antibody, where the linker molecule comprises at least one avidin moiety or at least one streptavidin moiety, or a combination of both,
(d) adding an amplification reagent under conditions such that the amplification reagent binds to the linker molecule, where the amplification reagent comprises (i) a polynucleotide molecule and (ii) at least one biotin moiety,
(e) performing polymerase chain reaction (PCR) on the resulting product of step (d) in the presence of a signal molecule, and
(f) detecting a PCR product produced in step (e) by detecting a signal from the signal molecule, thereby detecting PrP^{SC} in said sample.
   Biomolecule

The specific biomolecules to be detected in the methods of the present invention are not particularly limited. The methods may be used to detect any biomolecule that may be present at low levels in a test sample. Exemplary molecules that may be detected using the methods of the present invention include protein and peptides, glycoproteins, lipids, carbohydrates, nucleic acids, or any combination thereof.

In particular, the methods of the present invention are useful in the detection and/or monitoring of biomolecules such as viral antigens and prions that may be present in a biological sample, and biowarfare reagents such as ricin and botulinum toxin that may be present in an environmental sample or a biological sample.

More particularly, the methods of the present invention are useful in the detection and/or monitoring of prion in a biological fluid, such as PrP^{C} or PrP^{SC}, even more particularly, human PrP^{SC}, deer PrP^{SC}, or bovine PrP^{SC}.

Test Sample

The test sample used in the methods of the present invention may be from any source that may contain a selected biomolecule of interest. When the methods are used for the detection of biomolecules from living organisms, such as in the detection of a viral antigen, a prion or a toxin, preferably the source of the test sample is a biological fluid, such as plasma, serum, saliva, whole blood, semen, cerebrospinal fluid or urine, neuronal tissue, sputum, nasal material or bronchial secretions. Nasal material can include mucosal secretions from the anterior nasal passage or sinuses (secretions).

When the methods are used for the detecting of PrP^{SC}, the source of the test sample is any in which PrP^{SC} may be found at a detectable level using the method. For example, plasma, serum, saliva, whole blood, semen, and cerebrospinal fluid may be used. Preferably, the sample is plasma or serum.

When the methods are used for the detection of biomolecules in environmental sample, such as in the detection of toxins, the source of the test sample may be water, soil, air or any biological material.

The biological sample may be used directly isolated from a subject. Alternatively, the sample may be frozen to preserve it prior to use in the assay, or stored refrigerated for about two weeks. The sample may also be enriched or concentrated, such as by a molecular sizing column, filtration, or centrifugation, in order to increase the sensitivity of the assay. For example, when bovine PrP^{SC} is to be detected, the sample may be enriched for bovine PrP^{SC} prior to incubation of the sample with a capture molecule. Enrichment may be performed, for example, by adding of ribolyser to the sample to release from bovine PrP^{SC} in the sample. Enriching may also be performed by centrifugation or filtration of the sample, or by digesting the sample with Protease K.

The sample may also be fractionated, for example to remove molecules that may interfere with the detection of the selected biomolecule; supplemented, for example to add factors, such as blocking reagents (e.g. BSA, casein, triton X, polymers, or nucleic acids) that increase the ability of the capture molecule and the detector molecule to recognize and/or bind the selected biomolecule; and/or subjected to column purification, filtration, centrifugation, dialysis, lysis, and/or enzymatic digestion

Support

The solid support may be any structure that provides a support for the capture molecule. Preferably, the solid support is polystyrene, derivatized polystyrene, a membrane, such as nitrocellulose, PVDF or nylon, a latex bead, a glass bead, a silica bead, paramagnetic or latex microsphere, or microtiter well. As a further example, the solid support may be a modified microtiter plate, such as a Top Yield plate, which allows for covalent attachment of a capture molecule, such as an antibody, to the plate. When the solid support is a material such as a bead, paramagnetic microsphere or latex microsphere, the solid support may be contained in an open container, such as a multi-well tissue culture dish, or in a sealed container, such as a screw-top tube, both of which are commonly used in laboratories.

The solid support may be modified to facilitate binding of the capture molecule to the surface of the support, such as by coating the surface with poly L-lysine, or siliconized with amino aldehyde silane or epoxysilane. The skilled artisan will understand that the circumstances under which the methods of the current invention are performed will govern which solid supports are most preferred and whether a container is used.

In a preferred embodiment, the solid support is a paramagnetic microsphere which contains surface modifications for efficient conjugation of the capture molecule. Preferably, the paramagnetic microsphere is composed of Fe₂O₃ or Fe₃O₄ particles coated with a polymer (polystyrene) shell. Preferably, the size of the paramagnetic microsphere is about 50 nm to about 4.5 um, more preferably about 1-3 um. Preferably, the paramagnetic microsphere is coated with Protein A, Protein G, carboxylate or amine modifications to aid in the attachment of capture molecule to the surface of the microspheres.

Capture Molecule and Detector Molecule

The methods of the present invention may be adapted for the detection of any biomolecule by simply altering the capture molecule and the biomolecule-binding portion of the detector molecule used in the method (e.g., the capture antibody attached to the solid support and the biotinylated detector antibody) such that the capture molecule and the biomolecule-binding portion of the detector molecules utilized specifically recognize and bind the biomolecule for which the method is being used.

The capture molecule and the biomolecule-binding portion of the detector molecule may recognized and bind the same or different portions or epitopes of the biomolecule under investigation. Preferably, the capture molecule and the biomolecule-binding portion of the detector molecule recognize and bind different portions or epitopes of the biomolecule.

The specific molecules used as the capture molecule and the biomolecule-binding portion of the detector molecules used in the methods of the present invention are not particularly limited. Molecules useful as the capture molecule and the biomolecule-binding portion of the detector molecules include monoclonal, polyclonal, or phage derived antibodies, antibody fragments, peptides, ligands, haptens, nucleic acids, nucleic acid aptamers, protein A, protein G, folate, folate binding proteins, plasminogen, maleimide and sulfhydryl reactive groups, and those that may be produced for use with the methods of the present invention.

Preferably, the capture molecule and the biomolecule-binding portion of the detector molecules are monoclonal, polyclonal, or phage derived antibodies, or antibody fragments. More preferably, the capture molecule and the biomolecule-binding portion of the detector molecules are monoclonal antibodies.

Any method for generating monoclonal antibodies, for example by in vitro generation with phage display technology and in vivo generation by immunizing animals, such as mice, can be used in the present invention. These methods include the immunological methods described by Kohler and Milstein (Nature 256, 495-497 (1975)) and Campbell (“Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam (1995)); as well as by the recombinant DNA method described by Huse et al. (Science 246, 1275-1281 (1989)). Standard recombinant DNA techniques are described in Sambrook et al. (“Molecular Cloning,” Second Edition, Cold Spring Harbor Laboratory Press (1987)) and Ausubel (“Current Protocols in Molecular Biology,” Green Publishing Associates/Wiley-Interscience, New York (1990)). Each of these methods is incorporated herein by reference.

Other binding molecules include functional antibody equivalents that have binding characteristics that are comparable to those of the antibodies, and include, for example, chimerized, humanized, and single-chain antibodies as well as fragments thereof may also be used. Methods of producing such functional equivalents are disclosed in PCT Application WO 93/21319, European Patent Application No. 239,400; PCT Application WO 89/09622; European Patent Application 338,745; and European Patent Application EP 332,424. Each of these methods is incorporated herein by reference.

The capture molecule and the biomolecule-binding portion of the detector molecule are not limited to intact antibodies, but encompass other binding molecules such as antibody fragments and recombinant fusion proteins comprising an antibody fragment.

As used herein, “antibody fragments” include any portion of an antibody that retains the ability to bind to the epitope recognized by the full length antibody, generally termed “epitope-binding fragments.” Examples of antibody fragments preferably include, but are not limited to, Fab, Fab′, and F(ab′)₂, Fd, single-chain Fvs (scFv), single-chain antibodies, disulfide-linked Fvs (sdFv) and fragments comprising either a V_{L} or V_{H} domain. Epitope-binding fragments, including single-chain antibodies, may comprise the variable region(s) alone or in combination with the entirety or a portion of the following: hinge region, C_{H}1, C_{H}2, and C_{H}3 domains.

The antibodies and binding molecules of the present invention should have a high affinity for the selected biomolecule under investigation. Preferably, when antibodies are used as the capture molecule and in the detector molecules, they will have an affinity of 10⁻⁶-10⁻¹⁰/M, more preferably they will have an affinity of at least 10⁻⁸/M, most preferably they will have an affinity at least 10⁻⁹/M.

Exemplary capture molecules for use in the detection of PrP^{SC} in a test sample include the anti-PrP^{SC} monoclonal antibody 3F4 (Signet Pathology Systems, Dedham, Mass.); and 6H4 (Prionics AG, Zurich, Switzerland). Attention may be given to the use of a PrP^{sc}-specific antibody, peptide, plasminogen or ligand to discriminate between PrP^{sc} and PrP^{c} forms of prions.

Quantities of the capture molecule to be attached to the solid support may be determined empirically by checkerboard titration with different quantities of biomolecule that would be expected to mimic quantities in a test sample. Generally, the quantity of the biomolecule in the test sample is expected to be in the femtogram to milligram range. An unknown concentration of the biomolecule (e.g., PrP^{SC}) in a test sample will be added at specified volumes, and this will influence the sensitivity of the test. If large volumes of the test sample (e.g., 200-400 uL) are used, modification of the test format may be needed to allow for the larger sample volumes. Generally, however, the concentration of the capture molecule will be about 2 to about 5 micrograms per mL.

The capture molecule can be attached to a solid support by routine methods that have been described for attachment of biomolecules to plastic or other solid support systems (e.g., membranes or microspheres). Examples of such methods may be found in U.S. Pat. No. 4,045,384 and U.S. Pat. No. 4,046,723, both of which are incorporated herein by reference.

Conjugation should be optimized to ensure stabilization of the colloidal suspension, maintenance of the binding ability of the capture molecule, and minimization of surface interaction with other molecules present in the test reaction.

Attachment of the capture molecule to surfaces such as membranes, microspheres, or microtiter wells may be performed by direct addition in PBS, or other buffers of defined pH, followed by drying in a convection oven.

The capture molecule may be attached to the solid support by an attachment means, such as via adsorption, covalent linkage, avidin-biotin linkage, streptavidin-biotin linkage, heterobifunctional cross-linker, Protein A linkage or Protein G linkage. Each of the attachment means should permit the use of stringent washing conditions with minimal loss of the capture molecule from the surface of the solid support. As an example, the adsorption may be hydrophilic adsorption. As a further example, the heterobifunctional cross-linker may be maleic anhydride, 3-aminopropyl trimethoxysilane (APS), N-5 azido, 2-nitrobenzoyaloxysuccinimide (ANB-NOS) or mercaptosilane.

The capture molecule may be attached to the solid support though a portion of the capture molecule, such as amino acid residue, preferably a lysine or arginine residue, a thiol group or a carbohydrate residue. When the capture molecule is an antibody, the thiol group may be a thiol group of the antibody hinge region.

The solid support may be derivatized with avidin or streptavidin, and the capture molecule may be modified to contain at least one biotin moiety, to aid in the attachment of the capture molecule to the solid support. Alternatively, the solid support may be derivatized with biotin, and the capture molecule may be modified to contain at least one avidin or at least one streptavidin moiety.

In practicing the methods of the present invention, a test sample suspected of containing the selected biomolecule under investigation is applied to the support containing the capture molecule. Depending on the identity of the support, the support may be contained within a culture device of some type. When the support is a membrane, for example, a shallow glass dish slightly bigger that the length and width of the membrane may be used. When the support is a microsphere, the microspheres may be contained in a tube, such as a polypropylene or polystyrene screw-top tube. The identity of the container is not critical, but it should be constructed of a material to which the reagents used in the methods of the present invention do not adhere.

The quantity of test sample used is not critical, but should be an amount that can be easily handled and that has a concentration of biomolecule that is detectable within the limits of the methods of the present invention. The test sample should also be sufficient to adequately cover the support, and may be diluted if needed in this regard. For example, the quantity of the test sample may be between 5 uL and 2 mL. Preferably, the quantity of the test sample is be between 5 uL and 1 mL. Most preferably, the quantity of the test sample may be between 5 uL and 200 uL.

While the concentration of the biomolecule in the test sample is not critical, it should be within the detection limits of the methods of the present invention. The skilled artisan will understand that the concentration may vary depending on the volume of the test sample, and thus it is difficult to provide a concentration range over which a biomolecule may be detected. Preferably a test sample used in the methods contains between about 1×10⁻⁶ g and about 1×10⁻¹⁸ g of the biomolecule, more preferably between about 1×10⁻⁶ g and about 1×10⁻¹⁵ g of the biomolecule, most preferably between about 1×10⁻⁶ g and about 1×10⁻¹² g of the biomolecule.

The use of the reagents and methods taught herein allows the detection of biomolecules present in a sample at concentrations as low as the attogram (10⁻¹⁸) range. The methods and kits taught herein can thus be used to detect biomolecules present in a sample at a concentration of, for example, about 10 ng/mL or less, about 1 ng/mL or less, about 0.7 ng/mL or less, about 0.5 ng/mL or less, about 0.1 ng/mL or less, about 0.01 ng/mL or less, about 1 pg/mL or less, about 0.1 pg/mL or less, about 0.01 pg/mL or less, about 1 fg/mL or less, or about 1 ag/mL.

The length of time during which the capture molecule-bearing support is incubated with the test sample is not critical. Preferably, the incubation proceeds from between about 10 minutes and about 60 minutes, but may require overnight. More preferably, the incubation proceeds from between about 10 minutes and about 30 minutes. Most preferably, the incubation proceeds from between about 10 minutes and about 15 minutes.

The temperature at which each of the incubation steps of the methods is performed is also not critical. Preferably, the temperature at which the incubations occur is between about 18° C. and about 37° C. More preferably, the incubation temperature is between about 18° C. and about 30° C. Most preferably, the incubation temperature is at ambient temperature (20° C.).

While each of the incubation steps of the present invention can take place in a fixed, stationary position, it is preferable that the incubation steps occur under gentle agitation, rocking or shaking. Such movement ensures proper mixing and exposure of all of the elements used in the method. For example, when paramagnetic microspheres are used as the support, the test sample and reagents can be combined in a microtiter well and mixed on a magnetocapture platform, such as the Bionor Platform (Bionor Corp, Norway). The Bionor Platform is small (20 cm×30 cm), easily carried, and can be connected to a 12V car battery for portability.

Biotinylation of Detector Molecules

Following the incubation of the capture molecule-bearing support and the test sample, a detector molecule is added to the assay system under conditions that allow the detector molecule to recognize and bind the selected biomolecule that is bound by the capture molecule, which in turn, is attached to the solid support.

The detector molecule is comprised of two parts, (i) a molecule that specifically binds to the selected biomolecule, and (ii) at least one biotin moiety.

As described above, the portion of the detector molecule that specifically binds to the selected biomolecule is not critical and includes monoclonal, polyclonal, or phage derived antibodies, antibody fragments, peptides, ligands, haptens, nucleic acids, nucleic acid aptamers, protein A, protein G, folate, folate binding proteins, plasminogen, maleimide and sulfhydryl reactive groups, both those commercially available and those that may be produced for use with the methods of the present invention. Preferably, the biomolecule-binding portion of the detector molecule is a monoclonal, polyclonal, or phage derived antibody, or antibody fragment. More preferably, the biomolecule-binding portion of the detector molecule is a monoclonal antibody or antibody fragment.

The biotinylation of the detector molecule may be by routine methods (Altin et al. Anal Biochem. 224:382-389 (1995), incorporated herein by reference) or through the use of a commercial biotinylation kit, such as EZ-NHS-LC-biotin (Pierce Biotechnology Inc., Rockford, Ill., USA). Preferably, at least 1-4 biotin molecules are incorporated per nucleotide of the detector molecule when a polynucleotide is used. More preferably, at least 3-4 biotin molecules are incorporated per detector molecule.

Biotinylated detector molecules may be purified by dialysis against PBS using a molecular weight exclusion of 10,000 kD. The extent of detector molecule biotinylation may be determined in the final preparation using an avidin-HABA reagent to determine the molar ratio of biotin to detector molecule (Green N. M. Biochem J. 94:32c-24c (1965), incorporated herein by reference).

After the biotinylated detector molecule has been prepared, it is added to the assay system. The amount of biotinylated detector molecule added to the assay system depends on the quantity of the test sample used, and the surface area of the support. Thus the skilled artisan would understand that the concentration of biotinylated detector molecule added to the assay system will depend on the quantities of other elements already added. However, it is preferable that a quantity of biotinylated detector molecule between 0.1 and 1 times the amount of the capture molecule attached to the solid support be added. More preferably, a quantity of biotinylated detector molecule between 0.5 and 1 times the amount of the capture molecule attached to the solid support. Most preferably, a quantity of biotinylated detector molecule between 0.1 and 0.25 times the amount of the capture molecule attached to the solid support.

The length of time during which the detector molecule is incubated in the assay system is not critical. Preferably, the incubation proceeds from between about 10 minutes and about 60 minutes. More preferably, the incubation proceeds from between about 10 minutes and about 30 minutes. Most preferably, the incubation proceeds from between about 10 minutes and about 15 minutes.

Linker Molecule

The linker molecule used in the methods of the present invention serves as a bridge between the biotinylated detector molecule and the amplification reagent. The linker molecule is preferably avidin or streptavidin, or polymerized avidin or streptavidin.

When avidin is used as the linker molecule, the formation of a bridge between the biotinylated detector molecule and the amplification reagent is dependent on the molar ratio of avidin to biotin. To obtain the highest sensitivity and the lowest non-specific background, due to the potential of avidin to bind to other reagents or the solid support, the amount of avidin may be varied, starting with a molar excess and subsequently lowering the amount, in the manner reported by Saito et al., 1999 (Saito et al. Clin. Chem. 45(5):665-669 (1999); see also methods of determining the concentration of avidin to biotin as described by Niemeyer et al. Nuc. Acid Res. 27(23):4553-4561 (1999); both references are incorporated herein by reference). Assessment may also be performed using different concentrations of the amplification reagent in a matrix design. The exact cause of any background from avidin may be assessed by eliminating one reagent at a time, and noting unexpected signal.

The source of avidin is not particularly limited, and may be commercially obtained such as through Sigma-Aldrich Corp. or Pierce Biotechnology Inc.

The length of time during which the linker molecule is incubated in the assay system is not critical. Preferably, the incubation proceeds from between about 10 minutes and about 60 minutes. More preferably, the incubation proceeds from between about 10 minutes and about 30 minutes. Most preferably, the incubation proceeds from between about 10 minutes and about 15 minutes.

Amplification Reagent

An important feature of the invention is the ability to easily and quickly detect the presence of very small amounts of a biomolecule in a test sample. The speed and ease of the method are based in part on the use of the polymerase chain reaction (PCR).

The amplification reagent is comprised of two elements, (i) a polynucleotide molecule and (ii) at least one biotin moiety. Where the detector molecule is attached directly to the amplification reagent, a biotin moiety is not needed.

The polynucleotide molecule serves as a platform for the hybridization of a signal molecule. As the number of polynucleotide molecules increases throughout the course of PCR, so too does the amount of activated signal molecule bound to the PCR product. The identity of the polynucleotide molecule employed is not critical. Preferred examples of the polynucleotide molecule include synthetic or natural, organic or inorganic polymers of nucleic acids, such as DNA and RNA.

The polynucleotide may be a linear polynucleotide, such as a linear DNA (bio-DNA) or RNA molecule, or a circular polynucleotide, such as a circular DNA or RNA molecule. Preferably, the polynucleotide molecule is a DNA polynucleotide, a RNA polynucleotide or a peptide nucleic acid (PNA) polynucleotide.

The size of the polynucleotide molecule is also not critical, but should be large enough to serve as a platform for binding by the signal molecule. When a DNA polynucleotide is used as the scaffold molecule, the length of the polynucleotide may be between about 50 nucleotides and about 500 nucleotides, preferably between about 250 and about 500 nucleotides, most preferably greater than 250 nucleotides.

One useful polynucleotide molecule that has been found to be effective in the methods of the present invention is a 500 bp lambda bacteriophage sequence (SEQ ID NO:1: gatgagttcgtgtccgtacaactggcgtaatcatggcccttcggggccattgtttctctgtggaggagtccatgacgaaagatgaactgattg cccgtctccgctcgctgggtgaacaactgaaccgtgatgtcagcctgacggggacgaaagaagaactggcgctccgtgtggcagagct gaaagaggagcttgatgacacggatgaaactgccggtcaggacacccctctcagccgggaaaatgtgctgaccggacatgaaaatgag gtgggatcagcgcagccggataccgtgattctggatacgtctgaactggtcacggtcgtggcactggtgaagctgcatactgatgcacttc acgccacgcgggatgaacctgtggcatttgtgctgccgggaacggcgtttcgtgtctctgccggtgtggcagccgaaatgacagagcgc ggcctggccagaatgcaataacgggaggcgctgtggctgatttcgataacc).

The biotin moiety on the amplification reagent is capable of binding to the immobilized linker molecule. While the skilled artisan will understand how to biotinylate molecules such as polynucleotides, reference is made to Altin et al. (Anal Biochem. 224:382-389 (1995)), which is hereby incorporated by reference. Biotinylation kits are also available from commercial sources, such as the PCR Biotinylation Kit (KPL, Inc., Gaithersburg, Md.). Preferably, at least about 1% to about 50% of the nucleotides of the polynucleotide molecule are biotinylated, more preferably at least about 10% and about 30%, most preferably at least 25%.

The quantity of amplification reagent added to the assay system will be dependent on the quantity of the reagents added to the assay system in the previous steps. In general however, the quantity of amplification reagent added is such that the amount of biotin on the amplification reagent is at least 3 fold excess over the amount of linker molecule (avidin) previously added. Since each avidin is a tetrameric molecule capable of binding 4 biotin, in a preferred example, the quantity of amplification reagent added to the assay system is between about 3× and 12× fold excess, more preferably between about 3× and 9× fold excess, and most preferably between about 3× and 6× fold excess.

The amount of the amplification reagent used in assay methods can severely affect background (Constantine et al. Ultra-low detection of HIV p24 antigen using immuno-PCR. Abstract, XIV Inter. AIDS Conf. 2002, Barcelona, Spain, Jul. 7-12, 2002), resulting in a low signal to noise ratio. Assessment of the background may be performed using serial dilutions of the amplification reagent at concentrations of 1 pg/ml to 1 ng/ml, followed by assessment of the highest signal to noise ratio. Subsequently, modifications to this concentration may be performed after optimization of all other reagents, if necessary, such as performed by Niemeyer et al. (1999), incorporated herein by reference. Equally important for optimal amplification and signal/noise is the molar ratio of biotin to the scaffold molecule, and this may be assessed empirically, as mentioned above.

The length of time during which the amplification reagent is incubated in the assay system is not critical. Preferably, the incubation proceeds from between about 10 minutes and about 60 minutes. More preferably, the incubation proceeds from between about 10 minutes and about 30 minutes. Most preferably, the incubation proceeds from between about 10 minutes and about 15 minutes.

Signal Molecule

The signal molecule of the present invention comprises an oligonucleotide molecule that specifically binds the polynucleotide molecule portion of the amplification reagent. As the products of the PCR reaction are identical to at least portions of the polynucleotide molecules of the amplification reagent, the oligonucleotide molecule also specifically binds to products of the PCR. The signal molecule further comprises two moieties linked to the oligonucleotide molecule: (1) a fluorophore and (2) a quencher dye.

The fluorophore portion of the signal molecule (which upon excitation emits detectable fluorescence) is attached at the 5′ end of the oligonucleotide molecule. The quencher dye portion of the signal molecule (which prevents emission of fluorescence by the fluorophore when in close proximity to the fluorophore) is located at the 3′ end of the oligonucleotide molecule. During the combined annealing/extension phase of PCR, signal molecule is cleaved by the 5′-3′ exonuclease activity of the Taq DNA polymerase, separating the fluorophore and the quencher dye, and allowing the emission of fluorescence by the 5′ fluorophore. The signal molecule binds to the polynucleotide molecule portion of the amplification reagent by hydrogen bonding of complementary base nucleotides. It does not prime the template DNA, nor does it interfere with PCR (DNA polymerization). This results in detectable fluorescence that is proportional to the amount of accumulated PCR product.

The identity of the oligonucleotide molecule portion of the signal molecule will depend on the identity of the polynucleotide molecule portion of the amplification reagent. Preferably, the oligonucleotide molecule is a sequence that is the complement of a portion of the polynucleotide molecule. The oligonucleotide molecule may be either an exact complement of a portion of the polynucleotide molecule, or have 99%, 98%, 97%, 96%, 95%, 90%, 85%, or 80% homology with a complement of a portion of the polynucleotide molecule.

The length of the oligonucleotide molecule portion of the signal molecule may be between 1 and 100 nucleotides, preferably between 10 and 50 nucleotides.

The signal molecule of the present invention allows a quantitative or semi-quantitative determination of the amount of the biomolecule (e.g., PrP^{SC}) in a test sample, i.e., fluorescence.

The quantity of the signal molecule added to the assay system will depend on the quantity of the reagents added in the previous steps. In general however, the quantity of signal molecule added is such that there is at least a ten-fold molecular excess over the amplification reagent. In a preferred example, the quantity of signal molecule added to the assay system is between about 10 and 100 fold excess, more preferably between about 50 and 100 fold excess, and most preferably between about 50 and 75 fold excess.

The length of time during which the signal molecule is incubated in the assay system is not critical. Preferably, the incubation proceeds from between about 10 minutes and about 60 minutes. More preferably, the incubation proceeds from between about 10 minutes and about 30 minutes. Most preferably, the incubation proceeds from between about 10 minutes and about 15 minutes.

The detection of the signal molecule may be performed during or after incubation of the signal molecule in the assay system. If a substrate is required to be added to the assay system in order to detect the signal molecule, the instructions accompanying the substrate will be followed.

An alternative method for practicing the invention involves the use of a fluorescent molecule that selectively binds to a double-stranded polynucleotide. When such a fluorescent molecule is present during PCR, it is incorporated into the double-stranded polynucleotide produced during PCR. As the fluorescent signal is only detected in the presence of double-stranded polynucleotide, the amount of fluorescent signal is directly proportional to the amount of PCR product in a reaction vessel. Thus, a semi-quantitative or quantitative determination of the amount of PCR product in a sample can be made.

Other alternative methods for detecting the product of the amplification reaction include the use of an intercalating dye, such as SYBR Green, during the amplification reaction. In such a modification of the method, the intercalating dye replaces the use of the signal molecule.

Moreover, a chemiluminescent or luminescent signal can be used in place of the fluorophore and quencher on the signal molecule. The use of such signals involves the labeling of amplification (e.g., PCR) products with alkaline phosphatase and detection of the products via chemiluminescent signal. The amplification reaction can be carried out in the presence of biotinylated (bio-UTP or bio-TTP) precursors. The amplification reaction products can them be labeled by alkaline phosphatase (AP) by incubation with streptavidin labeled AP. Conversely, the amplification reaction products can be labeled with digoxigenin by use of DIG-11-dUTP or dTTP in place of UTP in the amplification reaction. The amplification reaction products are then incubated with anti-DIG labeled alkaline phosphatase. A chemiluminescent substrate such as CDP or CSPD can then be added. Non-reactive molecules are be washed away, and the specific signal detected by a chemiluminescent detector or exposure to chemiluminescent sensitive film.

In addition to the activation of the fluorophore upon separation from the quencher dye, due to the cleavage of the oligonucleotide molecule, the fluorophore can be separated from the quencher dye by the opening of hairpin structures, such as occurs through the use of Molecular Beacons, Scorpion and Amplifluor probes.

Fluorophore

The fluorophore that may be used in the signal molecule of the present invention is not particularly limited, but should be one that emits fluorescence between 400-700 nm. Preferred fluorophores include 6-carboxyfluorescein, Alexa, Cy, Texas Red, TET, HEX, JOE, Cys-3, VIC, ROX, Rhodamine and FAM. Preferably, the fluorophore is 6-carboxyfluorescein.

Quencher Dye

The quencher dye that may be used in the signal molecule of the present invention is not particularly limited, but should be one that absorbs fluorescence between 475-580 nm. Preferred quencher dyes include 3′-Black Hole Quencher dye, DABCL, TAMRA and QSY. Preferably, the quencher dye is 3′-Black Hole Quencher dye. Marras et al., Nucleic Acids Research 30(21):1-8 (2002), herein incorporated by reference, provides the efficiency of energy transfer for a large number of combinations of fluorophores and quenchers.

Polymerase Chain Reaction

After addition of the amplification reagent and the signal molecule, an amplification reaction is performed. Preferably, the amplification reaction is the polymerase chain (PCR), performed using an oligonucleotide primer or primers that is specific for the polynucleotide molecule portion of the amplification reagent (and/or compliment thereof). PCR may be conducted directly on the assay system in a microwell plate, or in some other suitable container (such as when microbeads are used as the support).

Preferably, after the amplification reagent and the signal molecule are added, a pre-PCR block is performed. Such a block involves adding a pre-PCR blocking reagent composed of a “DNA Blocking Reagent” (Roche Diagnostics; Indianapolis, Ind.) combined 1:1 with “Stabilcoat” (Surmodics, Eden Prairie, Minn.) to the assay system. Preferably, the block is performed for about one hour, and at room temperature.

After the block is performed, PCR amplification buffer, an oligonucleotide primer or primers specific to the polynucleotide molecule of the amplification molecule (and/or compliment thereof) and DNA polymerase are added. The skilled artisan will understand that the amplification buffer and DNA polymerase used in the PCR may vary depending on the nature and identity of the polynucleotide molecule portion of the amplification molecule and the nature and identity of the primer or primers. An exemplary PCR amplification buffer/primer/DNA polymerase composition is 10 mM Tris HCl, pH 8.3, 50 mM KCl, 4 mM MgCl₂, 0.2 mM each dNTP; 0.1 uM primer; 0.3 units of Platinum Taq DNA polymerase (Invitrogen, Carlsbad, Calif.). The skilled artisan will understand that many other DNA polymerases are available that may be used in the methods of the present invention.

The PCR amplification may be carried out in any of the commercially available systems for performing PCT. The time and temperature of the PCR will depend on the nature and identity of the polynucleotide molecule portion of the amplification molecule and the nature and identity of the primer or primers. Exemplary conditions include 20 cycles at 94° C., 68° C., 50 cycles at 94° C., 68° C.

Preferably, PCR is conducted twice. For example, after a number of cycles, an aliquot is removed from the reaction vessel and placed into a new vessel. Additional PCR reagents (amplification buffer, primer or primers, and DNA polymerase) are added, and the PCR thus repeated. A different, second PCR primer or primers may also be used. Repeating the PCR in a fresh vessel aids in the reduction of background noise.

For example, after 20 cycles of the first PCR reaction, 5 uL of the sample may be removed from the reaction mix and transferred to 20 uL fresh PCR reagents in standard PCR tubes. PCR amplification may then be continued for a second round of 50 cycles.

In addition to PCR amplification, the methods of the invention may be practiced using Strand Displacement Amplification (SDA), Rolling Circle Amplification (RCA), Transcription Mediated Amplification (TMA) or Ligase Chain Reaction (LCR). Amplification of signal may be generated in a homogeneous, closed tube environment, using Real-Time amplification. Instrumentation suitable for Real-Time amplification includes the ABI PRISM TaqMan system, Roche LightCycler, Idaho Technologies RapidCycler, Bio-Rad iCycler and Cepheid SmartCycler.

Detection of PCR Products

The signal molecule is activated during PCR such that the fluorophore portion of the signal molecule emits fluorescence. During the PCR, the signal molecule is hydrolyzed and thus the quencher molecule is separated from the fluorophore, thereby unmasking of the fluorophore.

Fluorescence may be detected by the iCycler real-time PCR instrument (Bio-Rad Laboratories; Hercules, Calif.) or other instruments suitable for detection of fluorescence. Detection of the signal may mediated by hybridization of probes relying on fluorescence resonance energy transfer (FRET)

The detection of the activated signal molecule includes real-time detection (see Sims et al., Anal. Biochem 281:230-232 (2000); McKie et al., J. Immunol. Methods 261:167-175 (2002)), and this, along with the other modifications, contribute to the excellent results disclosed herein.

Attachment of Detector Molecule to the Amplification Reagent

Reducing the number of reagents that have the potential to non-specifically bind to other reagents or to the solid support may be helpful. Thus, in each embodiment of the present invention, one or more of the reagents can be combined into one conjugate.

For example, (i) the portion of the detector molecule that binds to the biomolecule and (ii) the linker molecule can be joined prior to addition to the assay system. Thus, rather then using a biotinylated detector molecule and an avidin or streptavidin molecule, the detector molecule may be avidinylated. An example of such a conjugate would be a monoclonal antibody with at least one avidin moiety.

Another example is a conjugate comprising (i) the portion of the detector molecule that binds to the biomolecule and (ii) the amplification reagent. Such a conjugate could be formed using the chemical methods of Hendrickson et al. (Nuc. Acid Res. 23(3):522-529 (1995), incorporated herein by reference). The amplification reagent may be conjugated to the portion of the detector molecule that binds to the biomolecule by other methods described in the literature. For example, a standard procedure is one in which a linker molecule (e.g., Sulfo-SMCC) is attached to both a detector antibody and an amplification reagent (Hendrickson et al., 1995). The detector molecule may also be attached directly to the amplification reagent through covalently attachment, or the use of a heterobifunctional reagent, such carbodiimide EDC or a sulfhydryl reactive site. Other combinations will be apparent to the skilled artisan.

Blocking Solutions

A variety of commercially available solutions may be used as blocking reagents in the method described herein including BSA, heterogeneous nucleic acid, prionex (or any commercially available blocking reagent), casein, gelatin, collagen, PVP, PVA, serum, a cell lysate, non-fat dry milk, a non-ionic surfactant (such as Tween 20 or NP-40), heparin, a chelating agent, EDTA and Triton X-100. Non-specific reactivity may be blocked or reduced by using a blocking reagent. In each method of the present invention, blocking solution is added as the diluents for each reagent in the assay. They are removed by aspiration after specific binding of reagents to their targets has occurred. A preferred general blocking reagent is a 1:1 mixture of DNA Blocking Reagent (Roche Diagnostics, catalog number 1096176) and Stabilguard (Surmodics Corp., product number SG01-1000). A preferred blocking reagent for addition to the secondary antibody dilution buffer (to produce a 10% secondary antibody blocking reagent) is FcR Blocking Reagent (Miltenyi Corp., material number 120-000-442). Other blocking reagents may be obtained from commercial sources such as Pierce Superblock, Roche Stabilcoat, FcR blocking reagent, or blocking reagents from Boehringer Manheim, KPL or other vendors.

Blocking reagent may be added after any or all of the steps in the methods of the present invention. Preferably, blocking reagent is added after addition of the linker molecule and prior to PCR. Preferably, the blocking reagent used prior to PCR is 10 mg/ml gamma globulin-free BSA (Sigma Corp., catalog number A7030) diluted in DIAPOPS buffer (12.7 g Tris-HCl, 2.4 g Tris base, 8.8 g NaCl, 1.0 ml Tween-20, water to 100 ml; adjust pH to 7.5 with 1M NaOH; dilute 1:10 in water for use).

Washing Solution

Between the addition of reagents in the methods of the present invention, the assay system is preferably subjected to washing to reduce the incidence of non-specific binding.

While the number of wash cycles and soak times is empirically determined, in general either water or a low or high molarity salt solution with a detergent such as Tween 20, Triton X-100, or NP-40 may be used as the washing solution. 1-3 washes, each lasting 5-10 minutes may be performed, after incubation of each of the reagents used in the methods. Preferably, washing takes place between each incubation step, e.g., after addition of the capture molecule to the solid support, after addition of the test sample, after addition of the detector molecule, after addition of the linker molecule, after addition of the amplification reagent, and after addition of the linker molecule-signal molecule conjugate.

Preferably, the washing solution is PBS with 0.05% Tween 20, and the assay system is subjected to 2-3 washes between incubation steps, each wash lasting about 5 minutes. More preferably, 10 mM EDTA and 5 units/mL sodium heparin is added to all buffers and washing solutions.

In a preferred embodiment, the incubation and washing steps are performed using a platform that gently rocks a microtiter well containing the mixture of sample and reagents. For example, when paramagnetic microspheres are used as the support, the test sample and reagents can be combined in a microtiter well and mixed on a magnetocapture platform, such as the Bionor Platform (Bionor Corp, Norway). The Bionor Platform is small (20 cm×30 cm), easily carried, and can be connected to a 12V car battery for portability. It also contains a pump for aspiration of wash fluid and a reading lamp.

When a membrane is used, the test sample, membrane, and other reagents can be combined sequentially in a microtiter well, trough, or plastic housing, and gently rocked on the Bionor platform.

Kit

The present invention also includes a kit that may be used to detect a biomolecule in a test sample.

In one embodiment, the present invention relates to a kit for detecting a biomolecule in a sample comprising:
(a) a capture molecule attached to a solid support, wherein said capture molecule comprises a molecule that specifically binds to pre-selected biomolecule,
(b) a detector molecule, wherein said detector molecule comprises (i) a molecule that specifically binds to said pre-selected biomolecule and (ii) at least one biotin moiety,
(c) an linker molecule, wherein said linker molecule comprises at least one avidin moiety or at least one streptavidin moiety, or a combination thereof,
(d) an amplification reagent, wherein said amplification reagent comprises (i) a polynucleotide molecule and (ii) at least one biotin moiety,
(e) an oligonucleotide primer that specifically binds to said polynucleotide molecule,
(f) a signal molecule comprising (i) an oligonucleotide molecule that specifically binds the polynucleotide molecule, (ii) a fluorophore and (iii) a quencher dye, and
(g) a set of standards, wherein said standards allow an approximation of concentration of said pre-selected biomolecule in a sample.

In further embodiment, the present invention relates to a kit for detecting a biomolecule in a sample comprising:
(a) a first capture molecule attached to a solid support, wherein said first capture molecule comprises a molecule that specifically binds to a pre-selected biomolecule,
(b) a second capture molecule attached to said solid support, wherein said second capture molecule comprises a molecule that specifically binds to an internal control molecule,
(c) an internal control molecule,
(d) a first detector molecule-amplification reagent conjugate, said conjugate comprising a first detector molecule attached to a first polynucleotide molecule, wherein said first detector molecule comprises a molecule that specifically binds to said pre-selected biomolecule,
(e) a second detector molecule-amplification reagent conjugate, said conjugate comprising a second detector molecule attached to a second polynucleotide molecule, wherein said second detector molecule comprises a molecule that specifically binds to said internal control molecule, and wherein said second polynucleotide molecule is different from said first polynucleotide molecule,
(f) a first oligonucleotide primer that specifically binds to said first polynucleotide molecule,
(g) a second oligonucleotide primer that specifically binds to said second polynucleotide molecule,
(h) a first signal molecule comprising (i) an oligonucleotide molecule that specifically binds the first polynucleotide molecule, (ii) a first fluorophore and (iii) a quencher dye,
(i) a second signal molecule comprising (i) an oligonucleotide molecule that specifically binds the second polynucleotide molecule, (ii) a second fluorophore that can be distinguish from said first fluorophore and (iii) a quencher dye,
(j) a first set of standards, wherein said first set of standards allows an approximation of concentration of said pre-selected biomolecule in a sample, and
(k) a second set of standards, wherein said second set of standards allows an approximation of concentration of said internal control molecule in a sample.

In another embodiment, the present invention relates to a kit for detecting a biomolecule in a sample comprising:
(a) a capture molecule attached to a solid support, wherein said capture molecule comprises a molecule that specifically binds to a prion,
(b) a detector molecule, wherein said detector molecule comprises (i) a molecule that specifically binds to a prion, and (ii) at least one biotin moiety,
(c) an linker molecule, wherein said linker molecule comprises at least one avidin moiety or at least one streptavidin moiety, or a combination thereof,
(d) an amplification reagent, wherein said amplification reagent comprises (i) a polynucleotide molecule and (ii) at least one biotin moiety,
(e) an oligonucleotide primer that specifically binds to said polynucleotide molecule,
(f) a signal molecule comprising (i) an oligonucleotide molecule that specifically binds the polynucleotide molecule, (ii) a fluorophore and (iii) a quencher dye, and
(g) a set of standards, wherein said standards allow an approximation of concentration of prion in a sample between about 3 attograms and 3 nanograms.

In a preferred embodiment, the prion in the kit is PrP^{C} or PrP^{SC}.

In yet another embodiment, the present invention relates to a kit for detecting a biomolecule in a sample comprising:
(a) a capture molecule attached to a solid support, wherein said capture molecule comprises a molecule that specifically binds to a prion,
(b) a detector molecule, wherein said detector molecule comprises (i) a molecule that specifically binds to a prion, and (ii) at least one avidin moiety,
(c) an amplification reagent, wherein said amplification reagent comprises (i) a polynucleotide molecule and (ii) at least one biotin moiety,
(d) an oligonucleotide primer that specifically binds to said polynucleotide molecule,
(e) a signal molecule comprising (i) an oligonucleotide molecule that specifically binds the polynucleotide molecule, (ii) a fluorophore and (iii) a quencher dye, and
(f) a set of standards, wherein said standards allow an approximation of concentration of prion in a sample between about 3 attograms and 3 nanograms.

In a preferred embodiment, the prion in the kit is PrP^{C} or PrP^{SC}.

The identity and properties of each of the reagents used in the kits are the same as those defined above for the methods of the present invention. In a preferred embodiment, blocking, buffer, and wash solutions may also be included in the kit.

With regard to the standards used in the kits for membrane and solid supports, calibration may be performed to correlate signal development with prion copy number to allow a semi-quantitative measurement of prion. These standards will be run simultaneously to act as calibrators to insure a dosel response and linearity.

The internal control molecule is one that is mixed with a sample prior to use of the kit. This control allows the user to determine whether the components of the kit are active, and whether they have used the kit correctly, by producing a positive signal that can be distinguished from the signal that indicates the presence of the pre-selected biomolecule in the sample.

The kits of the present invention may also include written instructions for use of the kit. The kits may also include other components to aid in the detection of the selected biomolecule. The identity of these other components will be obvious to the skilled artisan based on the identity of the biomolecule that is the subject of the detection.

### EXAMPLES

The broad scope of this invention is best understood with reference to the following examples, which are not intended to limit the invention to specific embodiments.

### Example 1

Protocol

TopYield stripwell plates (NalgeNunc Corp., Naperville, Ill.) were coated with anti-prion antibody 8B4 (from Dr. Man Sun Sy, Case Western Reserve, Cleveland, Ohio) (capture antibody) in bicarbonate buffer (pH 9.4) for 3 hr, RT, and then blocked with Stabilcoat (Surmodics: Eden Prairie, Minn.) for 1 hr, at room temperature (RT). Varying concentrations of recombinant hamster prion protein (Prionics AG, Schieren, Switzerland) diluted in 0.1% Triton-X/PBS buffer were added to wells for 2 hr, RT. Plates were washed 6 times and then incubated with the detector antibody, a biotinylated 3F4 antibody (Signet Pathology Systems Inc, Dedham Mass.) (2.5 ug/mL) diluted in diluent buffer (Zeptometrix Corp., Buffalo, N.Y.), for 1 hr, RT. Plates were washed 6 times and then incubated with streptavidin (10 ng/mL) diluted in Zeptometrix diluent buffer for 1 hr, RT. Plates were washed 6 times and then blocked for 30 min with DNA Blocking Reagent (Roche Diagnostics; Indianapolis, Ind.).

Plates were then incubated with 1-10 pg/mL biotinylated reporter DNA amplification reagent: a 500 bp sequence from bacteriophage lambda (Edelman et al., BioTechniques 35(2):368-375 (2003))) (SEQ ID NO:1: gatgagttcgtgtccgtacaactggcgtaatcatg gcccttcggggccattgtttctctgtggaggagtccatgacgaaagatgaactgattgcccgtctccgctcgctgggtgaacaactgaacc gtgatgtcagcctgacggggacgaaagaagaactggcgctccgtgtggcagagctgaaagaggagcttgatgacacggatgaaactgc cggtcaggacacccctctcagccgggaaaatgtgctgaccggacatgaaaatgaggtgggatcagcgcagccggataccgtgattctg gatacgtctgaactggtcacggtcgtggcactggtgaagctgcatactgatgcacttcacgccacgcgggatgaacctgtggcatttgtgct gccgggaacggcgtttcgtgtctctgccggtgtggcagccgaaatgacagagcgcggcctggccagaatgcaataacgggaggcgct gtggctgatttcgataacc) diluted in DNA Blocking Reagent (Roche Diagnostics Corp, Indianapolis, Ind.) for 1 hour, RT. Wells were washed as follows: 3 washes, one 15 minute soak, 3 washes, and a final 5 minute soak with water. A pre-PCR block was performed (10 mg/mL BSA in wash buffer) for 1 hr at RT, followed by addition of 33 uL of PCR amplification buffer (10 mM Tris HCl, pH 8.3, 50 mM KCl, 4 mM MgCl₂, 0.2 mM each dNTP, 0.1 uM each primer (5′-GATGAGTTCGTGTCCGTACAACTGG-3′ (SEQ ID NO:2) and 5′-GGTTATCGAAATCAGCACAGCGCC-3′ (SEQ ID NO:3)) 0.3 units of Platinum Taq DNA polymerase (Invitrogen, Carlsbad, Calif.).

Amplification was carried out directly in the TopYield strip wells in the iCycler with real-time measurements of signal using either SYBR Green 1 (1:50,000) or the BHQ-1 probe (120 nM) by the iCyclerIQ™ Multi-Color Real Time PCR Detection System (Bio-Rad Laboratories, Hercules, Calif.). After 20 cycles, 5 uL was removed from the reaction mix and transferred to 29 uL new PCR reagents in standard PCR tubes. PCR amplification was continued for a second round of 50 cycles.

Fluorescence was detected during PCR amplification by hydrolysis of the signal molecule that was labeled with a 5′-reporter dye: 6-carboxyfluorescein, and a 3′-quencher dye: 3′-Black Hole Quencher dye (Biosearch Technologies, Inc. Novato, Calif.).

Results

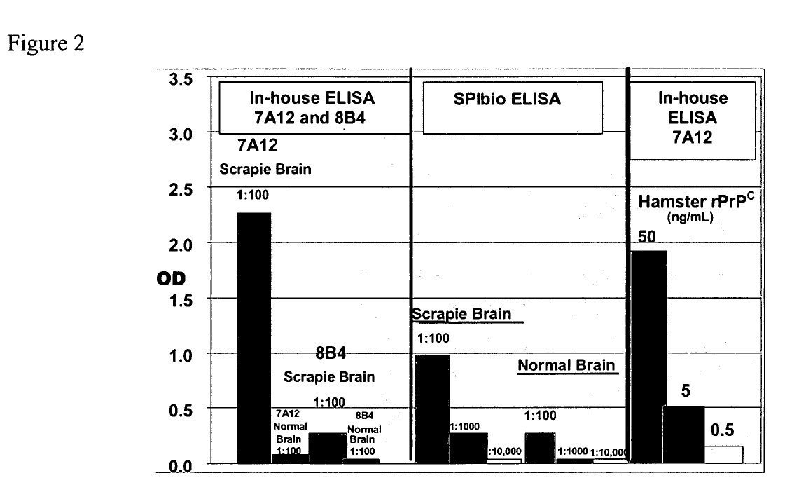

To compare the sensitivity of detection of the method of the present invention to other serological methods, the ELISA test (SPIbio Corp, Massy Cedex, France) was performed on the kit positive and negative controls, standard recombinant hamster PrP, and dilutions of Proteinase K (PK) treated normal and scrapie brain homogenates (FIG. 2). The ELISA test was able to detect the PK treated scrapie brain homogenate at a 1:100 dilution using 8B4 and at a 1:1000 dilution using 7A12 (data not shown). It could be estimated that the amount of PrP^{SC} present in the 1:100 dilution of the Proteinase K treated scrapie brain homogenate was approximately 5-50 ng/mL (which was the limit of detection for the standard recombinant hamster PrP). Therefore, the ELISA limit of sensitivity of detection for PrP^{SC} is down to a 1:100-1:1000 dilution of scrapie brain homogenate (depending upon the specific antibody used) which approximates 5-50 ng/mL of PrP^{SC}.

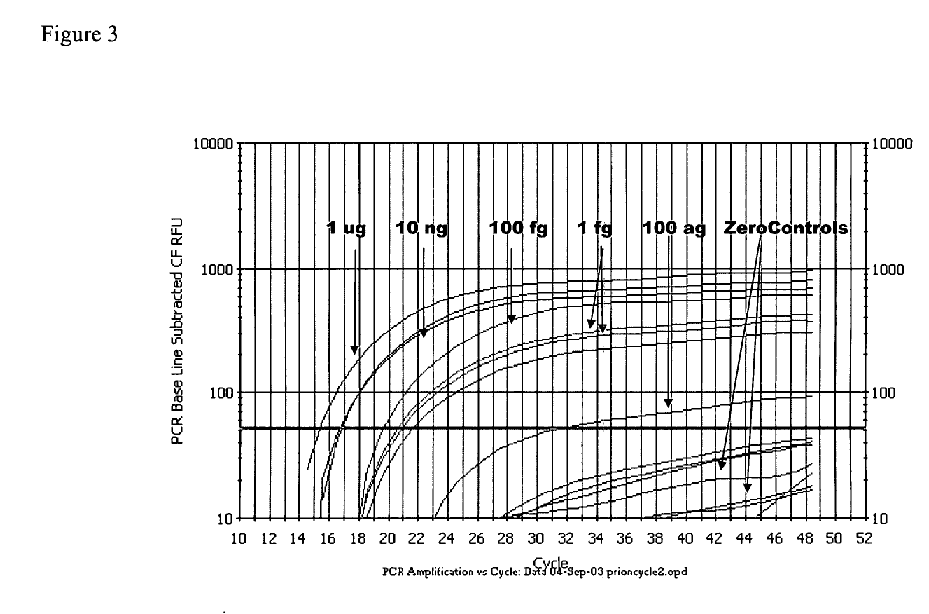

FIG. 3 shows the Immuno-PCR (IPCR) results using the methods of the present invention for the detection of recombinant hamster prion protein. IPCR detected levels as low as 100 ag/mL by real-time measurements, with subsequent runs consistently detecting 1 fg/mL (data not shown). Results exhibited good reproducibility, a linear dose response curve (average 4 Cts per log), a dynamic range from 1 ug/mL to 100 ag/mL, and a sensitivity that outdistances all other methods for the detection of prion protein.

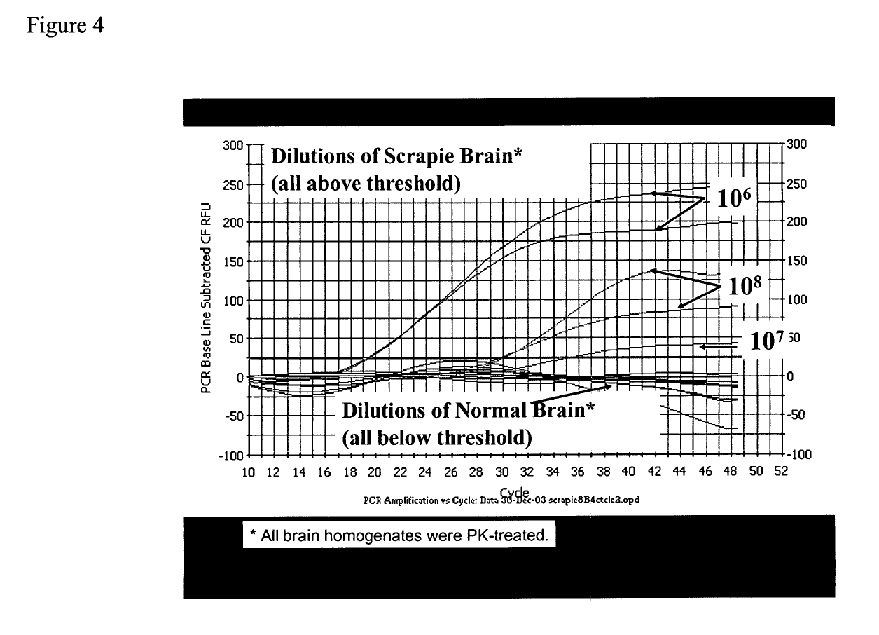

FIG. 4 shows the IPCR for the detection of PK treated scrapie and normal brain homogenates diluted down to 1:10⁸, a dilution which is 6 logs lower (10,000× more dilute) than the dilution (1:100) detected by the standard ELISA method.

Modifications

Several critical approaches may be used in conjunction with this system to reduce background and ensuring reproducibility of low level detection have been incorporated into the methods of the present invention. These refinements:

1. Use of the same animal species for the capture and detector molecule, with specific regard to use of antibodies as the capture and detector molecules (to decrease animal interspecies non-specific cross reactivity).

2. Use of a DNA:Stabilcoat pre-PCR blocking reagent composed of a “DNA Blocking Reagent” (Roche Diagnostics; Indianapolis, Ind.) combined 1:1 with “Stabilcoat” (Surmodics, Eden Prairie, Minn.) designed to minimize non-specific protein and DNA interactions in nucleic acid hybridizations (Roche Diagnostics; Indianapolis, Ind.).

3. Addition of “FcR Blocking Reagent” (Miltenyi Biotec; Auburn, Calif.) to the secondary antibody dilution to block antibody Fc receptor non-specific interactions, when the capture and detector molecules are antibodies.

4. Addition of 10 mM EDTA and 5 Units/mL sodium heparin to all buffers and wash reagents to minimize non-specific binding caused by molecular ionic interactions,

5. Addition of 2 extra blocking steps (one after the linker is added) with DNA:Stabilcoat blocking reagent and pre-PCR (described by Nunc-Nalge Corp. in a TechNote described on the Nalgenunc.com website in paper DP0031.

6. A larger DNA reporter template (polynucleotide molecule portion of the amplification reagent) (500 bp) than is standard for use in probe hydrolysis (signal molecule) protocols (where the reporter template is recommended to be in the range of 100-150 bp). The DNA reporter template is biotinylated throughout the template (approximately 25% of all nucleotides in the sequence) allowing increased probability of binding to streptavidin. In routine IPCR protocols, the DNA reporter template possesses a single biotin only at the 5′ end of the molecule.

7. Washing of TopYield strips after addition of amplification reagent using an automated washer with 96 individual probes for each well to decrease crossover contamination between wells.

8. A volume reduction of the PCR reaction volume in the TopYield wells to 33 uL and the introduction of a 2-step amplification cycle. The first step of amplification is performed for 20 cycles, at which time a 5 uL aliquot is removed and transferred to a new PCR reagent mix (20 uL) in regular PCR tubes for another round of 50 cycles. This particular step is a major contribution to the enhancement of higher signal/low background ratio.

### Example 2

### Method for the Detection of a Biomolecule in a Test Sample

A. Sample Collection, Processing and Storage:

This method may be used with biological samples such as any human body fluid (e.g., whole blood, plasma, serum, saliva, neuronal tissues, and urine) or environmental samples such as water or soil.

All specimens used in this assay may be processed on the same day as collected or stored frozen at −20° C. or below until tested. Clear, non-hemolyzed plasma or serum specimens should be used whenever possible.

B. Test Protocol:

Note: All steps require optimization depending on the antigen or antibody.

1. The solid support (e.g., microwells, microbeads, or membranes) is coated with antibody solution (5-10 ug/mL) for 8-16 hrs at RT.

2. The solution is removed by aspiration and the blocking reagent is added for 1 hr at RT.

3. The solution is removed by aspiration and the sample (10-100 uL) is added for 10 min to 1 hr at RT.

4. The solid support is washed 1-4× using PBS/detergent and 10-100 uL detector antibody (2-5 ug/mL) in diluent buffer is added for 10 min to 1 hr at RT.

5. The solid support is washed 1-4× using PBS/detergent and 10-100 uL linker (10-100 ng/mL) in diluent buffer is added for 10 min to 1 hr at RT.

6. The solid support is washed 1-4× using PBS/detergent and 10-100 uL amplification reagent (10-100 pg/mL) in diluent buffer is added for 10 min to 1 hr at RT.

7. The solid support is washed 1-4× using PBS/detergent and 100-300 uL pre-PCR blocking reagent (1:1 ratio of DNA Blocking Reagent (Roche Diagnostics; Indianapolis, Ind.) and Stabilcoat (Surmodics, Eden Prairie, Minn.)) is added for 10 min to 1 hr at RT.

8. The solid support is washed 1-4× using PBS/detergent and 10-50 uL of PCR amplification buffer (containing 10 mM Tris HCl, pH 8.3, 50 mM KCl, 4 mM MgCl₂, 0.2 mM each dNTP), 0.1 uM primer, 0.3 units of DNA polymerase and 10-100 uL signal molecule (100-200 nM) in diluent buffer is added for 10 min to 1 hr at RT.

9. PCR is performed under the following cycling conditions. An initial 6 min cycle at 95° C., followed by 20 cycles of 1 min at 95° C. and 2 min at 68° C. in TopYield strips. 5 uL from each reaction was then aliquoted into new PCR reagents for the second amplification of 50 cycles using the same times and temperatures.

10. Fluorescence produced by activated signal molecule is detected by the iCycler iQTM Multi-Color Real Time PCR Detection System (Bio-Rad Laboratories, Hercules, Calif.).

11. The end product may be compared to a set of standards and quantified.

While the invention has been described in detail and with reference to specific embodiments thereof, it will be apparent to one of skill in the art that various changes and modifications can be made therein without departing from the spirit and scope thereof.

1. A method for detecting a biomolecule in a sample, said method comprising:
(a) incubating a sample with a capture molecule that specifically binds a selected biomolecule under conditions such that said selected biomolecule in said sample is bound by said capture molecule, wherein said capture molecule comprises a molecule that specifically binds to said biomolecule and said capture molecule is attached to a solid support,
(b) incubating the resulting product of step (a) with a detector molecule that specifically binds said selected biomolecule under conditions such that said selected biomolecule bound by said capture molecule is also bound by said detector molecule, wherein said detector molecule comprises (i) a molecule that specifically binds to said biomolecule and (ii) at least one biotin moiety,
(c) incubating the resulting product of step (b) with a linker molecule under conditions such that said linker molecule binds to a biotin moiety of said detector molecule, wherein said linker molecule comprises at least one avidin moiety or at least one streptavidin moiety, or a combination thereof,
(d) incubating the resulting product of step (c) with an amplification reagent under conditions such that said amplification reagent binds to said linker molecule, wherein said amplification reagent comprises (i) a polynucleotide molecule and (ii) at least one biotin moiety,
(e) performing polymerase chain reaction (PCR) on the resulting product of step (d) in the presence of a signal molecule, and
(f) detecting a PCR product produced in step (e) by detecting a signal from the signal molecule, thereby detecting a biomolecule in said sample.

2. A method for detecting a biomolecule in a sample, said method comprising:
(a) incubating a sample with a capture molecule that specifically binds a selected biomolecule under conditions such that said selected biomolecule in said sample is bound by said capture molecule, wherein said capture molecule comprises a molecule that specifically binds to said biomolecule and said capture molecule is attached to a solid support,
(b) incubating the resulting product of step (a) with a detector molecule that specifically binds said selected biomolecule under conditions such that said selected biomolecule bound by said capture molecule is also bound by said detector molecule, wherein said detector molecule comprises (i) a molecule that specifically binds to said biomolecule and (ii) at least one avidin moiety or at least one streptavidin moiety, or a combination thereof,
(c) incubating the resulting product of step (b) with an amplification reagent under conditions such that said amplification reagent binds to said avidin or said streptavidin, or both, of said detector molecule, wherein said amplification reagent comprises (i) a polynucleotide molecule and (ii) at least one biotin moiety,
(d) performing polymerase chain reaction (PCR) on the resulting product of step (c) in the presence of a signal molecule, and
(e) detecting a PCR product produced in step (d) by detecting a signal from the signal molecule, thereby detecting a biomolecule in said sample.

3. A method for detecting a biomolecule in a sample, said method comprising:
(A) incubating a sample with a capture molecule that specifically binds a selected biomolecule under conditions such that said selected biomolecule in said sample is bound by said capture molecule, wherein said capture molecule comprises a molecule that specifically binds to said biomolecule and said capture molecule is attached to a solid support,
(B) incubating the resulting product of step (A) with a detector-signal conjugate, said conjugate comprising:
(i) a detector molecule comprising (a) a molecule that specifically binds to said biomolecule and (b) at least one biotin moiety,
(ii) a linker molecule bound to a biotin moiety of said detector molecule, wherein said linker molecule comprises at least one avidin moiety or at least one streptavidin moiety, or a combination thereof,
(iii) an amplification reagent bound to an avidin moiety or a streptavidin moiety, or both, of said linker molecule, wherein said amplification reagent comprises (a) a polynucleotide molecule and (b) at least one biotin moiety,
(C) performing polymerase chain reaction (PCR) on the resulting product of step (B) in the presence of a signal molecule, and
(D) detecting a PCR product produced in step (C) by detecting a signal from the signal molecule, thereby detecting a biomolecule in a sample.

4. A method for detecting a biomolecule in a sample, said method comprising:
(a) incubating a sample with a capture molecule that specifically binds a selected biomolecule under conditions such that said selected biomolecule in said sample is bound by said capture molecule, wherein said capture molecule comprises a molecule that specifically binds to said biomolecule and said capture molecule is attached to a solid support,
(b) incubating the resulting product of step (a) with a detector molecule-amplification reagent conjugate, said conjugate comprising a detector molecule attached to an amplification reagent, wherein said detector molecule comprises a molecule that specifically binds to said biomolecule and wherein said amplification reagent comprises a polynucleotide molecule,
(c) performing polymerase chain reaction (PCR) on the resulting product of step (b) in the presence of a signal molecule, and
(d) detecting a PCR product produced in step (c) by detecting a signal from the signal molecule, thereby detecting a biomolecule in a sample.

5. The method of claim 4, wherein the detector molecule is covalently attached to the amplification reagent.

6. The method of claim 4, wherein the detector molecule is attached to the amplification reagent by a heterobifunctional reagent.

7. The method of claim 6, wherein the heterobifunctional reagent is carbodiimide EDC or a sulfhydryl reactive site.

8. The method of any one of claims 1-4, wherein said capture molecule is attached to the solid support by an attachment means selected from the group consisting of adsorption, covalent linkage, avidin-biotin linkage, streptavidin-biotin linkage, heterobifunctional cross-linker, Protein A linkage and Protein G linkage.

9. The method of claim 8, wherein said adsorption is hydrophilic adsorption.

10. The method of claim 8, wherein the solid support is derivatized with avidin or streptavidin, and the capture molecule comprises at least one biotin moiety.

11. The method of claim 8, wherein the solid support is derivatized with biotin, and the capture molecule comprises at least one avidin moiety or at least one streptavidin moiety.

12. The method of claim 8, wherein the heterobifunctional cross-linker is selected from the group consisting of maleic anhydride, 3-aminopropyl trimethoxysilane (APS), N-5 azido, 2-nitrobenzoyaloxysuccinimide (ANB-NOS) and mercaptosilane.

13. The method of claim 8, wherein said capture molecule is attached to the solid support by a portion of the capture molecule selected from the group consisting of a lysine residue, an arginine residue, a thiol group and a carbohydrate residue.

14. The method of claim 13, wherein said capture molecule is an antibody and the thiol group is a thiol group of the antibody hinge region.

15. The method of any one of claims 1-4, further comprising blocking or reducing non-specific reactivity by adding a blocking reagent.

16. The method of claim 15, wherein the blocking reagent is selected from the group consisting of a non-ionic surfactant, heparin, a chelating agent, EDTA, BSA, serum, a cell lysate, and non-fat dry milk.

17. The method of claim 15, wherein the non-ionic surfactant is Tween-20 or NP 40.

18. The method of any one of claims 1-4, wherein said biomolecule is a polypeptide or a peptide.

19. The method of claim 18, wherein said polypeptide is a biological toxin selected from the group consisting of botulinum toxin and ricin.

20. The method of claim 18, wherein said polypeptide is a prion.

21. The method of claim 20, wherein said prion is human PrP^{SC}, deer PrP^{SC}, or bovine PrP^{SC}.

22. The method of any one of claims 1-4, wherein said sample is an environmental sample selected from the group consisting of water, soil, air and a biological material.

23. The method of any one of claims 1-4, wherein said sample is a biological sample selected from the group of consisting of serum, plasma, saliva, whole blood, semen, urine, cerebrospinal fluid, neuronal tissue, sputum, nasal secretions and bronchial secretions.

24. The method of any one of claims 1-4, wherein the capture molecule and the portion of the detector molecule that specifically binds to the biomolecule are each independently a member selected from the group consisting of a monoclonal antibody, a polyclonal antibody, a phage derived antibody, an antibody fragment, a peptide, a ligand, a hapten, a nucleic acid, a nucleic acid aptamer, protein A, protein G, folate, a folate binding protein, plasminogen, a maleimide reactive group and a sulfhydryl reactive group.

25. The method of claim 24, wherein said capture molecule and the portion of the detector molecule that specifically binds to the biomolecule are both a monoclonal antibody or antibody fragment.

26. The method of any one of claims 1-4, wherein said solid support is a member selected from the group consisting of polystyrene, derivatized polystyrene, a membrane, a latex bead, a glass bead, a silica bead, a paramagnetic microsphere, a latex microsphere, and a microtiter well.

27. The method of claim 26, wherein said solid support is a microtiter well.

28. The method of claim 26, wherein said membrane is selected from the group consisting of a nitrocellulose membrane, a PVDF membrane and a nylon membrane.

29. The method of any one of claims 1-4, wherein said polynucleotide molecule is a DNA polynucleotide, a RNA polynucleotide or a peptide nucleic acid polynucleotide.

30. The method of claim 29, wherein said polynucleotide molecule is a DNA polynucleotide.

31. The method of claim 29, wherein said polynucleotide molecule is a DNA polynucleotide of at least 250 nucleotides.

32. The method of any one of claims 1-3, wherein at least about 10% of nucleotides of said polynucleotide molecule are biotinylated.

33. The method of any one of claims 1-4, wherein the signal molecule comprises an oligonucleotide molecule that specifically binds a product of the PCR, and wherein said oligonucleotide molecule has a fluorophore and a quencher dye bound thereto.

34. The method of any one of claims 1-4, wherein the step of performing PCR is conducted in the presence of an oligonucleotide primer that hybridizes with said polynucleotide molecule and primes said PCR.

35. The method of claim 33, wherein the fluorophore is selected from the group consisting of 6-carboxyfluorescein, Alexa, Cy5, Texas Red, TET, HEX, JOE, Cys-3, VIC, ROX, Rhodamine and FAM.

36. The method of claim 33, wherein the fluorophore is 6-carboxyfluorescein.

37. The method of claim 33, wherein the quencher dye is selected from the group consisting of 3′-Black Hole Quencher dye, DABCL, TAMRA and QSY.

38. The method of claim 33, wherein the quencher dye is 3′-Black Hole Quencher dye.

39. The method of any one of claims 1-4, wherein said detecting a PCR product by detecting a signal from the signal molecule is performed by detecting a fluorescent signal.

40. The method of any one of claims 1-4, wherein the step of performing PCR is repeated.

41. The method of any one of claims 1-4, wherein the step of performing PCR is repeated using a second primer.

42. The method of any one of claims 1-4, wherein about 300 fg or less of said biomolecule is detected in said sample.

43. The method of any one of claims 1-4, wherein about 0.7 ng/mL or less of said biomolecule is detectable in said sample.

44. The method of any one of claims 1-4, wherein about 0.1 ng/mL or less of said biomolecule is detectable in said sample.

45. The method of any one of claims 1-4, wherein about 1 pg/mL or less of said biomolecule is detectable in said sample.

46. The method of any one of claims 1-4, wherein about 0.01 pg/mL or less of said biomolecule is detectable in said sample.

47. A method for detecting bovine PrP^{SC} in a sample, said method comprising:
(a) incubating a sample with a capture molecule that specifically binds bovine PrP^{SC} under conditions such that bovine PrP^{SC} in said sample is bound by said capture molecule, wherein said capture molecule comprises a molecule that specifically binds to bovine PrP^{SC} and said capture molecule is attached to a solid support,
(b) incubating the resulting product of step (a) with a detector molecule that specifically binds bovine PrP^{SC} under conditions such that bovine PrP^{SC} bound by said capture molecule is also bound by said detector molecule, wherein said detector molecule comprises (i) a molecule that specifically binds to bovine PrP^{SC} and (ii) at least one biotin moiety,
(c) incubating the resulting product of step (b) with a linker molecule under conditions such that said linker molecule binds to a biotin moiety of said detector molecule, wherein said linker molecule comprises at least one avidin moiety or at least one streptavidin moiety, or a combination thereof,
(d) incubating the resulting product of step (c) with an amplification reagent under conditions such that said amplification reagent binds to said linker molecule, wherein said amplification reagent comprises (i) a polynucleotide molecule and (ii) at least one biotin moiety,
(e) performing polymerase chain reaction (PCR) on the resulting product of step (d) in the presence of a signal molecule, and
(f) detecting a PCR product produced in step (e) by detecting a signal from the signal molecule, thereby detecting bovine PrP^{SC} in said sample.

48. The method of claim 47, further comprising enriching said sample for bovine PrP^{SC} prior to incubation of the sample with a capture molecule in (a).

49. The method of claim 48, wherein said enriching is performed by addition of ribolyser to said sample to release bovine PrP^{SC} in said sample.

50. The method of claim 48, wherein said enriching is performed by centrifugation or filtration of said sample.

51. The method of claim 48, wherein said enriching is performed by digestion of said sample with Protease K.

52. The method of claim 47, wherein said sample is a biological sample selected from the group of consisting of serum, plasma, saliva, whole blood, semen, urine, cerebrospinal fluid, neuronal tissue, sputum, nasal secretions and bronchial secretions.

53. The method of claim 47, wherein said solid support is a microtiter well.

54. The method of claim 47, wherein said polynucleotide molecule is a DNA polynucleotide.

55. The method of claim 47, wherein said polynucleotide molecule is a DNA polynucleotide of at least 250 nucleotides.

56. The method of claim 47, wherein at least about 10% of nucleotides of said polynucleotide molecule are biotinylated.

57. The method of claim 47, wherein the step of performing PCR is conducted in the presence of an oligonucleotide primer that hybridizes with said polynucleotide molecule and primes said PCR.

58. The method of claim 47, wherein the signal molecule comprises an oligonucleotide molecule that specifically binds a product of the PCR, and wherein said oligonucleotide molecule has a fluorophore and a quencher dye bound thereto.

59. The method of claim 47, wherein the fluorophore is selected from the group consisting of 6-carboxyfluorescein, Alexa, Cy5, Texas Red, TET, HEX, JOE, Cys-3, VIC, ROX, Rhodamine and FAM.

60. The method of claim 47, wherein the fluorophore is 6-carboxyfluorescein.

61. The method of claim 47, wherein the quencher dye is selected from the group consisting of 3′-Black Hole Quencher dye, DABCL, TAMRA and QSY.

62. The method of claim 47, wherein the quencher dye is 3′-Black Hole Quencher dye.

63. The method of claim 47, wherein said detecting a PCR product by detecting a signal from the signal molecule is performed by detecting a fluorescent signal.

64. The method of claim 47, wherein the step of performing PCR is repeated.

65. The method of claim 47, wherein about 300 fg or less of said bovine PrP^{SC} is detected in said sample.

66. The method of claim 47, wherein about 0.7 ng/mL or less of said bovine PrP^{SC} is detectable in said sample.

67. The method of claim 47, wherein about 0.1 ng/mL or less of said bovine PrP^{SC} is detectable in said sample.

68. The method of claim 47, wherein about 1 pg/mL or less of said bovine PrP^{SC} is detectable in said sample.

69. The method of claim 47, wherein about 0.01 pg/mL or less of said bovine PrP^{SC} is detectable in said sample.

70. A kit comprising:
(a) a capture molecule attached to a solid support, wherein said capture molecule comprises a molecule that specifically binds to a pre-selected biomolecule,
(b) a detector molecule, wherein said detector molecule comprises (i) a molecule that specifically binds to said pre-selected biomolecule and (ii) at least one biotin moiety,
(c) an linker molecule, wherein said linker molecule comprises at least one avidin moiety or at least one streptavidin moiety, or a combination thereof,
(d) an amplification reagent, wherein said amplification reagent comprises (i) a polynucleotide molecule and (ii) at least one biotin moiety,
(e) an oligonucleotide primer that specifically binds to said polynucleotide molecule,
(f) a signal molecule comprising (i) an oligonucleotide molecule that specifically binds the polynucleotide molecule, (ii) a fluorophore and (iii) a quencher dye, and
(g) a set of standards, wherein said standards allow an approximation of concentration of said pre-selected biomolecule in a sample.

71. The kit of claim 70, wherein said capture molecule and said detector molecule are each independently a member selected from the group consisting of a monoclonal antibody, a polyclonal antibody, a phage derived antibody, an antibody fragment, a peptide, a ligand, a hapten, a nucleic acid, a nucleic acid aptamer, protein A, protein G, folate, a folate binding protein, plasminogen, a maleimide reactive group and a sulfhydryl reactive group.

72. The kit of claim 70, wherein said capture molecule and the portion of the detector molecule that specifically binds to the biomolecule are both a monoclonal antibody or antibody fragment.

73. The kit of claim 70, wherein said solid support is a member selected from the group consisting of polystyrene, derivatized polystyrene, a membrane, a latex bead, a glass bead, a silica bead, a paramagnetic microsphere, a latex microsphere, and a microtiter well.

74. The kit of claim 73, wherein said solid support is a microtiter well.

75. The method of claim 73, wherein said membrane is selected from the group consisting of a nitrocellulose membrane, a PVDF membrane and a nylon membrane.

76. The kit of claim 70, wherein said polynucleotide molecule is a DNA polynucleotide, a RNA polynucleotide or a peptide nucleic acid polynucleotide.

77. The kit of claim 70, wherein said polynucleotide molecule is a DNA polynucleotide.

78. The kit of claim 70, wherein said polynucleotide molecule is a DNA polynucleotide of at least 250 nucleotides.

79. The kit of claim 70, wherein at least about 10% of nucleotides of said polynucleotide molecule are biotinylated.

80. The kit of claim 70, where said pre-selected biomolecule is a prion selected from the group consisting of human PrP^{SC}, bovine PrP^{SC}, or deer PrP^{SC}.

81. The kit of claim 70, wherein the fluorophore is selected from the group consisting of 6-carboxyfluorescein, Alexa, Cy5, Texas Red, TET, HEX, JOE, Cys-3, VIC, ROX, Rhodamine and FAM.

82. The kit of claim 70, wherein the fluorophore is 6-carboxyfluorescein.

83. The kit of claim 70, wherein the quencher dye is selected from the group consisting of 3′-Black Hole Quencher dye, DABCL, TAMRA and QSY.

84. The kit of claim 70, wherein the quencher dye is 3′-Black Hole Quencher dye.

85. The kit of claim 70, wherein said set of standards allows an approximation of concentration of said pre-selected biomolecule in said sample at a concentration of less than about 0.7 ng/mL.

86. The kit of claim 70, wherein said set of standards allows an approximation of concentration of said pre-selected biomolecule in said sample at a concentration of less than about 0.1 ng/mL.

87. The kit of claim 70, wherein said set of standards allows an approximation of concentration of said pre-selected biomolecule in said sample at a concentration of less than about 1 pg/mL.

88. The kit of claim 70, wherein said set of standards allows an approximation of concentration of said pre-selected biomolecule in said sample at a concentration of less than about 0.01 pg/mL.

89. A kit comprising:
(a) a first capture molecule attached to a solid support, wherein said first capture molecule comprises a molecule that specifically binds to a pre-selected biomolecule,
(b) a second capture molecule attached to said solid support, wherein said second capture molecule comprises a molecule that specifically binds to an internal control molecule,
(c) an internal control molecule,
(d) a first detector molecule-amplification reagent conjugate, said conjugate comprising a first detector molecule attached to a first polynucleotide molecule, wherein said first detector molecule comprises a molecule that specifically binds to said pre-selected biomolecule,
(e) a second detector molecule-amplification reagent conjugate, said conjugate comprising a second detector molecule attached to a second polynucleotide molecule, wherein said second detector molecule comprises a molecule that specifically binds to said internal control molecule, and wherein said second polynucleotide molecule is different from said first polynucleotide molecule,
(f) a first oligonucleotide primer that specifically binds to said first polynucleotide molecule,
(g) a second oligonucleotide primer that specifically binds to said second polynucleotide molecule,
(h) a first signal molecule comprising (i) an oligonucleotide molecule that specifically binds the first polynucleotide molecule, (ii) a first fluorophore and (iii) a quencher dye,
(i) a second signal molecule comprising (i) an oligonucleotide molecule that specifically binds the second polynucleotide molecule, (ii) a second fluorophore that can be distinguish from said first fluorophore and (iii) a quencher dye,
(j) a first set of standards, wherein said first set of standards allows an approximation of concentration of said pre-selected biomolecule in a sample, and
(k) a second set of standards, wherein said second set of standards allows an approximation of concentration of said internal control molecule in a sample.
